SEQUENCE LISTING (50 sequences)

SEQ ID NO: 1 - DNA, 400 bp, Nicotiana benthamiana
  source 1..400: mol_type = genomic DNA
gaatgacatcagcggaagcagtagtatatgctgcatcggcggcggcatgacagaatcattctcgccggaaacttcgccggcagagattacttcactgaaacgcctctctgaaacattggaatctatcttcgatgcggcttctccggagtttgactacttcgccgacgctaagcttgtgattcccggcgccggtaaggaaattccggttcaccggtgcattttgtcggcgaggagtccgttctttaagaatttgttctgcgggaaaaaggagaagaatagtaataaggtggaattaaaggaaataatgaaagagtatgaagtgagctatgatggtgtggtgagtgtgttggcctatttgtatagtggaaaaattaggccttcacctaaagatgtgtgtgtt

SEQ ID NO: 2 - DNA, 22 bp, synthetic construct
  source 1..22: mol_type = other DNA
tgtgtgtgtttgtgtggacaat

SEQ ID NO: 3 - DNA, 22 bp, synthetic construct
  source 1..22: mol_type = other DNA
tgtgtgtgtttgtgtggacaat

SEQ ID NO: 4 - DNA, 20 bp, synthetic construct
  source 1..20: mol_type = other DNA
gaacgctacagctggcctac

SEQ ID NO: 5 - DNA, 20 bp, synthetic construct
  source 1..20: mol_type = other DNA
ctggtacctcccaagctgac

SEQ ID NO: 6 - DNA, 157 bp, Nicotiana benthamiana
  source 1..157: mol_type = genomic DNA
accaaagcaagatcgttacgccctcagaacatcaccccagtggcttggccctcaaattgaggtcatccgttctgcaaccaagatgattgagagagagattaactcagtgaacgacaaccctttgatcgatgtttcaagaaacaaggcgttacacggt

SEQ ID NO: 7 - DNA, 20 bp, synthetic construct
  source 1..20: mol_type = other DNA
tgccatggcttcatactgtt

SEQ ID NO: 8 - DNA, 20 bp, synthetic construct
  source 1..20: mol_type = other DNA
cggcactttgtacgtggtta

SEQ ID NO: 9 - DNA, 153 bp, Nicotiana benthamiana
  source 1..153: mol_type = genomic DNA
ccagaggtcaatagaagcacttcaggccacaatatggcaggtttcctccgttcttatgagggtgcagaaaaaaatatctcgttcacatatactcgcgagtactcatgtcccgggtaaagcatcttgggaccaagctgttaagcgtgctttgca

SEQ ID NO: 10 - DNA, 20 bp, synthetic construct
  source 1..20: mol_type = other DNA
attccgccatctctgacttg

SEQ ID NO: 11 - DNA, 21 bp, synthetic construct
  source 1..21: mol_type = other DNA
ggcacctcaagacgaatgata

SEQ ID NO: 12 - DNA, 1371 bp, Nicotiana benthamiana
  source 1..1371: mol_type = genomic DNA
atgactactcacaaagctcattgcttgatcttgccatatccagcccaaggtcatatcaaccctatgctccaattctccaaacgtttgcaatccaaaggtgtgaaaatcactattgcagcgaccaaatccttcttgaaaaacatgcaagagttgtcaacttctgtgtcagtcgaggctatctccgatggctatgatgatggtggccgtgagcaagctggaacctttgtggcctattttacaagattcaaagaagttggctcggatactctgtctcagcttattgaaacgttaatgattcgtggttgtcctgtaaattgcattgtttacgatccatttcttccttgggctgtagaagtagcaaagaagtttggattagctactgctgcttttttcacacaatcttgtacagtggataacatttattaccatgtacataaaggggttctaaaacttcctccaactgatgttgatgaagaaatctcaattcctggattattaacagttgagacatcagatgtacctacttttgaaattaatcctgaatcaacaagagttcttgaaatgttggtgaatcagttctcgaatcttgataaagtggattgggttctaatcaacagcttctatgaattggagaaagaggtaattgattggatgtccgaggtttatccagtcaagacaattggaccagttataccatcaatgtacttagacaagaggctactagatgataaagaatatggccttagtgtcttcaaaccaatgacagatgcatgtctaaactggttaaaccatcaaccagctagctcagtagtatatgtatcatttggaagcttagccaaattagaagcagaacaaatggaagaagtagcatggggtttgagaaatagcaacaagaacttcttatgggtagttagatccactgaaaaatccaaacttcccaagaactttgtagaggaattagcaagtgaaaaaggcttagtggtgtcatggtgtccacaattacaagtcttggaacacaaatcaatcgggtgttttctcacgcactgtgggtggaattcgactttagaagcgattagtttgggagtaacaatggttgcgatgccgcaatggtcaggtcaacctacaaatgcaaagcttgtgaaggatatttgtgagatgggtgttagagcaaaacaagatgagaaagggattgttagaagagaaattattgaagaatatataaagatagtaatggaagaagagaaaggaaaaatgattaggaaaagtgcaaagaaatggaaggaattggctagaaaagctgtggatgaaggaggaagttcagatagaaatattgaagaatttgtttccaagttgatgactatttcttaatag

SEQ ID NO: 13 - DNA, 31 bp, synthetic construct
  source 1..31: mol_type = other DNA
cctctagaatgactactcacaaagctcattg

SEQ ID NO: 14 - DNA, 32 bp, synthetic construct
  source 1..32: mol_type = other DNA
tgagctcctattaagaaatagtcatcaacttg

SEQ ID NO: 15 - DNA, 25 bp, synthetic construct
  source 1..25: mol_type = other DNA
tggccagctagctatcactgaaaag

SEQ ID NO: 16 - DNA, 24 bp, synthetic construct
  source 1..24: mol_type = other DNA
cctggggtcagtcttatcttcagc

SEQ ID NO: 17 - DNA, 20 bp, synthetic construct
  source 1..20: mol_type = other DNA
ccttcgcaagacccttcctc

SEQ ID NO: 18 - DNA, 20 bp, synthetic construct
  source 1..20: mol_type = other DNA
ttgaaacatcgatcaaaggg

SEQ ID NO: 19 - DNA, 20 bp, synthetic construct
  source 1..20: mol_type = other DNA
ttggtcccaagatgctttac

SEQ ID NO: 20 - DNA, 23 bp, synthetic construct
  source 1..23: mol_type = other DNA
catttgaaggcagaggcgaacac

SEQ ID NO: 21 - DNA, 21 bp, synthetic construct
  source 1..21: mol_type = other DNA
actgagctagctggttgatgg

SEQ ID NO: 22 - RNA, 23 nt, synthetic construct
  source 1..23: mol_type = other RNA
tgcagatgttgctaagagagggg

SEQ ID NO: 23 - RNA, 23 nt, synthetic construct
  source 1..23: mol_type = other RNA
actgcagatgttgctaagagagg

SEQ ID NO: 24 - RNA, 23 nt, synthetic construct
  source 1..23: mol_type = other RNA
accgattcacgagcagaacttgg

SEQ ID NO: 25 - RNA, 23 nt, synthetic construct
  source 1..23: mol_type = other RNA
ggaggcaagaatactcccacgtc

SEQ ID NO: 26 - DNA, 23 bp, synthetic construct
  source 1..23: mol_type = other DNA
acgattggcgtgctatacgcagg

SEQ ID NO: 27 - RNA, 23 nt, synthetic construct
  source 1..23: mol_type = other RNA
ggaaactgctaaccgcacgatat

SEQ ID NO: 28 - DNA, 55 bp, synthetic construct
  source 1..55: mol_type = other DNA
acactctttccctacacgacgctcttccgatctctaatggtccattgcatatttc

SEQ ID NO: 29 - DNA, 55 bp, synthetic construct
  source 1..55: mol_type = other DNA
gtgactggagttcagacgtgtgctcttccgatctgtgcaagatctagaagttctg

SEQ ID NO: 30 - DNA, 54 bp, synthetic construct
  source 1..54: mol_type = other DNA
acactctttccctacacgacgctcttccgatctgttccacttcacatttccttc

SEQ ID NO: 31 - DNA, 24 bp, synthetic construct
  source 1..24: mol_type = other DNA
cagagcaacccctcttgtctgtac

SEQ ID NO: 32 - DNA, 24 bp, synthetic construct
  source 1..24: mol_type = other DNA
caaactaagtggaagatcatacag

SEQ ID NO: 33 - DNA, 24 bp, synthetic construct
  source 1..24: mol_type = other DNA
gtcattcaaggtaagtttctagtg

SEQ ID NO: 34 - DNA, 23 bp, synthetic construct
  source 1..23: mol_type = other DNA
cttatggaagatcgtatacagtg

SEQ ID NO: 35 - DNA, 22 bp, synthetic construct
  source 1..22: mol_type = other DNA
ctaatggtccattgcatatttc

SEQ ID NO: 36 - DNA, 21 bp, synthetic construct
  source 1..21: mol_type = other DNA
gttccacttcacatttccttc

SEQ ID NO: 37 - DNA, 55 bp, synthetic construct
  source 1..55: mol_type = other DNA
acactctttccctacacgacgctcttccgatctgtcgaagggcagctgctgattc

SEQ ID NO: 38 - DNA, 55 bp, synthetic construct
  source 1..55: mol_type = other DNA
gtgactggagttcagacgtgtgctcttccgatcttttagttgcccaaatcttcag

SEQ ID NO: 39 - DNA, 55 bp, synthetic construct
  source 1..55: mol_type = other DNA
acactctttccctacacgacgctcttccgatctggtcgaaggccagctgttgaac

SEQ ID NO: 40 - DNA, 57 bp, synthetic construct
  source 1..57: mol_type = other DNA
gtgactggagttcagacgtgtgctcttccgatctctacactaatttcatcatctatg

SEQ ID NO: 41 - DNA, 22 bp, synthetic construct
  source 1..22: mol_type = other DNA
gtcgaagggcagctgctgattc

SEQ ID NO: 42 - DNA, 21 bp, synthetic construct
  source 1..21: mol_type = other DNA
tttagttgcccaaatcttcag

SEQ ID NO: 43 - DNA, 22 bp, synthetic construct
  source 1..22: mol_type = other DNA
ggtcgaaggccagctgttgaac

SEQ ID NO: 44 - DNA, 23 bp, synthetic construct
  source 1..23: mol_type = other DNA
ctacactaatttcatcatctatg

SEQ ID NO: 45 - DNA, 300 bp, Nicotiana benthamiana
  source 1..300: mol_type = genomic DNA
gtttcttagttcctccacttctgcccaaaaacaagactcagagcgttcacctgtttgcaccctctcttcatgctctaaccatcgttgtgtgaacctataacgcttcggcctagccctgatcatgtaaggtcctgtatcttcattcttcaaatgcctgtaatagtttgcaatatccaagggctcaacttgcctgcggaactgcgtccctagttttatccattcctttcttccctcaaaactatctgggagctcataccttttcaacatttcaatgatttcgtcccatattcctgctagctc

SEQ ID NO: 46 - skipped

SEQ ID NO: 47 - RNA, 23 nt, synthetic construct
  source 1..23: mol_type = other RNA
gcaatggcatttgaagacaaggg

SEQ ID NO: 48 - RNA, 23 nt, synthetic construct
  source 1..23: mol_type = other RNA
cactggaaatgggaaactggtgg

SEQ ID NO: 49 - RNA, 23 nt, synthetic construct
  source 1..23: mol_type = other RNA
tatgctgcatgtaatctgaaagg

SEQ ID NO: 50 - RNA, 23 nt, synthetic construct
  source 1..23: mol_type = other RNA
atcccggaattatcagcacgagg

(19) United States
(12) Patent Application Publication
(10) Pub. No.: US 2026/0062711 A1
(43) Pub. Date: Mar. 5, 2026

(54) METHOD FOR PRODUCING DESIRED SUBSTANCE USING PLANT HAVING SUPPRESSED RESISTANCE

(71) Applicant: NATIONAL INSTITUTE OF ADVANCED INDUSTRIAL SCIENCE AND TECHNOLOGY, Tokyo (JP)

(72) Inventors: Noriho Fukuzawa, Tsukuba-shi (JP); Go Atsumi, Tsukuba-shi (JP); Yasushi Tasaka, Tsukuba-shi (JP); Takeshi Matsumura, Tsukuba-shi (JP)

(73) Assignee: NATIONAL INSTITUTE OF ADVANCED INDUSTRIAL SCIENCE AND TECHNOLOGY, Tokyo (JP)

(21) Appl. No.: 19/106,802

(22) PCT Filed: Sep. 1, 2023
(86) PCT No.: PCT/JP2023/032129

§ 371 Date: Feb. 26, 2025

(30) Foreign Application Priority Data
Sep. 5, 2022 (JP) 2022-140969

(51) Int. Cl. C12N 15/82; C07K 14/435

(52) U.S. Cl. CPC C12N 15/8205; C07K 14/43595; C12N 15/8241

(57) ABSTRACT

Provided is a method for achieving high expression of a target substance in a plant. Also provided are a method of creating a plant with suppressed resistance to a pathogen, wherein the method comprises suppression or disruption of expression of a gene associated with a resistance mechanism in the plant, and a method of creating a plant with suppressed resistance to a pathogen wherein the method comprises overexpression of a gene related to degradation of salicylic acid (SA) in plants; as well as a transgenic plant or genome-edited plant obtained by the method; and a method for producing a target protein using the plant.

### FIELD

The invention relates to a method for creating a plant with suppressed pathogen-induced resistance. The invention also provides transformed plant cells or a transgenic plant, or genome-edited plant, obtained by the aforementioned method. The invention further relates to a method for producing a target substance using the transformed plant cells or the transgenic plant, or genome-edited plant.

### BACKGROUND

Plants are exposed to various forms of stress such as variation in the growth environment, infection by pathogenic organisms, and feeding damage by insects or herbivores. Such stress leads to impaired plant growth, reducing agricultural harvest yields and leading to significant economic loss. To overcome such stress, plants have developed inherent self-defense mechanisms (resistance mechanisms or immune responses) which counter individual types of stress. For example, plants synthesize abscisic acid (ABA) when detecting changes in the environment (such as dryness, low temperature or high salt concentration), salicylic acid (SA) in response to infection by pathogenic organisms, and jasmonic acid (JA) in response to feeding damage by insects, and these self-defense mechanisms are regulated by a network of plant hormone signal transduction pathways.

Known mechanisms of resistance to pathogens include local resistance induced at sites of infection with pathogens, and accompanying systemic acquired resistance induced in tissues distant from infection sites. The former type of resistance is induced by recognition of pathogens by pattern recognition receptors (PRR) or nucleotide-binding domains and leucine-rich repeat receptors (NLR), which are found mainly in plants. The latter type of resistance is induced as a defense mechanism against secondary infection in tissues distant from infection sites, triggered by induction of local resistance. SA-mediated signal transduction plays an important role in either case.

With ongoing elucidation of plant self-defense mechanisms, much development has been carried out from the viewpoint of imparting greater resistance, such as reinforcing self-defense mechanisms, or creation of resistant variants by cross-breeding between related varieties or wild species. PTL 1, for example, discloses an AtPPR1 gene which improves blight resistance in plants, and PTL 2 discloses a method of creating transgenic plants that overproduce jasmonic acid (JA). PTL 3 discloses a method for down regulation of the Arabidopsis thaliana C protein phosphatase gene which functions as a negative regulator in the plant defense pathway. PTL 4 relates to a method for increasing SAR gene expression to augment disease resistance in a wide range.

However, since almost all transient expression systems generally utilize the infection and proliferation mechanisms of plant pathogens, attempting to produce a desired protein by transfer of an exogenous gene into a plant has been problematic in that the activation of self-defense mechanisms also suppresses expression of the exogenous gene, which may lower production of the desired protein depending on the type of host plant, the size of the exogenous gene, or the like.

NPL 1 describes creating recombinant plant species with suppression of genes related to gene silencing, which is a major function among plant resistance mechanisms, and reports that in studying target gene expression in the plants, recombinant plants with drastically reduced expression of the DCL2 and DCL4 genes compared to the wild type had higher expression levels of green fluorescent protein compared to the wild type plants.

### CITATION LIST

### Patent Literature

[PTL 1] U.S. Patent Application Publication No. 2021-0301299
[PTL 2] U.S. Patent Application Publication No. 2013-0111632
[PTL 3] U.S. Pat. No. 7,910,801
[PTL 4] International Patent Publication No. 00/53762

### Non Patent Literature

[NPL 1] “Demonstrated research and development on creating genetically modified plants in a closed plant factory (Project)—Technology evaluation report (final evaluation)”, (https://www.meti.go.jp/policy/tech_evaluation/c00/C0000000H28/161227_plant_factory_2nd/p lant_factory_2nd.html)

### SUMMARY

### Technical Problem

A need therefore exists for methods allowing high production of target substances in plants.

### Solution to Problem

As a result of much research directed toward meeting the aforementioned need, the present inventors have succeeded in achieving high expression of target proteins in plants with suppressed resistance mechanisms, based on the reverse idea of breaking down the self-defense mechanisms inherent in plants, which is an opposite approach to the conventional techniques of imparting resistance.

In other words, it was found that by suppressing or disrupting expression of a gene associated with a resistance mechanism in plants or overexpressing a gene associated with SA degradation in plants, it is possible to create a plant with a suppressed resistance mechanism to pathogens. It was found that the method described above can produce transformed plant cells or a transgenic plant (hereafter referred to as “a transgenic plant, etc.”) or a genome-edited plant, having suppressed or disrupted expression of a gene associated with a resistance mechanism, or a transgenic plant, etc., with overexpression of a gene associated with SA degradation. It was further found that it is possible to overexpress a target protein in such a transgenic plant, etc., or genome-edited plant since its resistance mechanism is not sufficiently activated therein, and the present invention was thus completed.

Specifically, the invention provides the following.

(1) A method for creating a plant with suppressed resistance to a pathogen, wherein the method comprises suppressing or disrupting expression of a gene associated with a resistance mechanism in the plant.

(2) The method according to (1), wherein the gene associated with a resistance mechanism is selected from genes associated with the salicylic acid (SA) biosynthetic pathway and genes associated with SA signal transduction.

(3) The method according to (2), wherein the gene associated with the SA biosynthetic pathway is selected from the phenylalanine ammonia-lyase (PAL) gene, isochorismate synthase (ICS) gene, EDS1 (enhanced disease susceptibility 1) gene, EDS5 (enhanced disease susceptibility 5) gene, Phytoalexin Deficient 4 (PAD4) gene, salicylate decarboxylase (SDC) gene, salicylic acid glucoside hydrolase gene, AIM1 (abnormal inflorescence meristem 1) gene, PBS3 (avrPphB susceptible 3) gene, EPS1 (enhanced pseudomonas susceptibility 1) gene, SARD1 (SAR-deficient 1) gene and calmodulin binding protein 60g (CBP60g).

(4) The method according to (2) or (3), wherein the gene associated with SA signal transduction is selected from an NPR1 (nonexpressor of pathogenesis-related genes 1) gene and an NPR3/4 gene.

(5) The method according to any one of (1) to (4), the method comprising:
(i) a step of preparing a nucleic acid construct for suppression or disruption of the gene associated with a resistance mechanism,
(ii) a step of transferring the nucleic acid construct into plant cells or plant tissue, and
(iii) a step of culturing the plant cells or plant tissue having the transferred nucleic acid construct to create a plant expressing the nucleic acid construct.

(6) The method according to (5), wherein the nucleic acid construct is a first nucleic acid construct containing a nucleic acid sequence complementary to mRNA of the gene associated with a resistance mechanism or a transcriptional product thereof.

(7) The method according to (6), wherein in the first nucleic acid construct, the nucleic acid sequence complementary to mRNA of the gene associated with a resistance mechanism or a transcriptional product thereof is selected from the group consisting of antisense RNA, RNA interference (RNAi) molecules, and virus-induced gene silencing (VIGS) molecules.

(8) The method according to (6) or (7), wherein the first nucleic acid construct has an expression promoter sequence that functions in the plant, a nucleic acid sequence complementary to mRNA of the gene associated with a resistance mechanism or a transcriptional product thereof, and an optionally used terminator sequence that functions in the plant, in that order.

(9) The method according to any one of (1) to (4), the method comprising:
(i) a step of preparing a second nucleic acid construct containing a nucleic acid sequence encoding a genome editing-related protein with a target site in the gene associated with a resistance mechanism,
(ii) a step of transferring the nucleic acid construct into plant cells or plant tissue, and
(iii) a step of culturing the plant cells or plant tissue having the transferred nucleic acid construct to create a plant with a mutation transferred into the gene sequence.

(10) The method according to (9), wherein the genome editing-related protein in the second nucleic acid construct contains a protein selected from the group consisting of a Cas protein, a zinc finger nuclease and a TAL effector nuclease.

(11) The method according to (10), wherein the genome editing-related protein in the second nucleic acid construct further contains a nucleic acid sequence-recognition module and/or guide RNA.

(12) The method according to any one of (9) to (11), wherein the second nucleic acid construct has an expression promoter sequence that functions in the plant, a nucleic acid sequence encoding the genome editing-related protein, and an optionally used terminator sequence that functions in the plant, in that order.

(13) The method according to any one of (5) to (8), wherein the transfer of the nucleic acid construct into the plant in step (ii) is carried out using a transient expression system.

(14) The method according to (13), wherein the transient expression system is selected from agroinfiltration, a plant viral vector and agroinfection that combines both.

(15) The method according to (14), wherein the plant viral vector is selected from the group consisting of full viral vectors, and deconstructed viral vectors such as tobacco mosaic virus (TMV), plum pox virus (PPV), turnip vein-clearing virus (TVCV), potato virus X (PVX), bean yellow dwarf virus (BEYDV), alfalfa mosaic virus (AIMV), cucumber mosaic virus (CMV), cowpea mosaic virus (CPMV), zucchini yellow mosaic virus (ZYMV), tobacco rattle virus (TRV), apple latent spherical virus (ACMV), bromo mosaic virus (BMV), tomato mosaic virus (ToMV), tomato yellow leaf curl virus (TYLCV) and tomato golden mosaic virus (TGMV).

(16) The method according to (14), wherein the agroinfection is a combination of CMV and Agrobacterium T-DNA.

(17) The method according to any one of (5) to (8), wherein the transfer of the nucleic acid construct into the plant in step (ii) is carried out by permeation or injection of a solution containing the nucleic acid construct into the plant.

(18) A method for creating a plant with suppressed resistance to a pathogen, wherein the method comprises overexpression of a gene related to salicylic acid (SA) degradation in the plant.

(19) The method according to (18), wherein the gene associated with SA degradation is selected from the group consisting of the salicylic acid glucosyltransferase (SGT) gene, UGT74F1, UGT74F2, UGT76B1 or UGT75B1 genes, salicylic acid 3-hydroxylase (S3H) gene, salicylic acid 5-hydroxylase (S5H) gene and salicylate hydroxylase (nahG) gene.

(20) The method according to (19), wherein the gene associated with SA degradation is the SGT gene.

(21) The method according to any one of (18) to (20), the method comprising:
(i) a step of preparing a nucleic acid construct for overexpression of the gene associated with SA degradation,
(ii) a step of transferring the nucleic acid construct into plant cells or plant tissue, and
(iii) a step of culturing the plant cells or plant tissue having the transferred nucleic acid construct to create a plant expressing the nucleic acid construct.

(22) The method according to (21), wherein the nucleic acid construct is a third nucleic acid construct containing a nucleic acid sequence of all or a portion of a gene associated with SA degradation.

(23) The method according to (21) or (22), wherein the third nucleic acid construct has an expression promoter sequence that functions in the plant, the nucleic acid sequence of all or a portion of the gene associated with SA degradation, and an optionally used terminator sequence that functions in the plant, in that order.

(24) The method according to any one of (21) to (23), wherein the transfer of the nucleic acid construct into the plant in step (ii) is carried out using a transient expression system.

(25) The method according to (24), wherein the transient expression system is selected from agroinfiltration, a plant viral vector and agroinfection that combines both.

(26) The method according to (25), wherein the plant viral vector is selected from the group consisting of full viral vectors, and deconstructed viral vectors such as tobacco mosaic virus (TMV), plum pox virus (PPV), turnip vein-clearing virus (TVCV), potato virus X (PVX), bean yellow dwarf virus (BEYDV), alfalfa mosaic virus (AIMV), cucumber mosaic virus (CMV), cowpea mosaic virus (CPMV), zucchini yellow mosaic virus (ZYMV), tobacco rattle virus (TRV), apple latent spherical virus (ACMV), bromo mosaic virus (BMV), tomato mosaic virus (ToMV), tomato yellow leaf curl virus (TYLCV) and tomato golden mosaic virus (TGMV).

(27) The method according to (25), wherein the agroinfection is a combination of CMV and Agrobacterium T-DNA.

(28) The method according to any one of (21) to (27), wherein the transfer of the nucleic acid construct into the plant in step (ii) is carried out by permeation or injection of a solution containing the nucleic acid construct into the plant.

(29) Transformed plant cells or a transgenic plant, or a genome-edited plant with suppressed or disrupted expression of a gene associated with a resistance mechanism, obtained by the method according to any one of (1) to (17).

(30) The transformed plant cells or transgenic plant, or genome-edited plant according to (29), wherein the gene associated with a resistance mechanism is an NPR1 gene.

(31) The transformed plant cells or transgenic plant, or genome-edited plant according to (29), wherein the gene associated with a resistance mechanism is an EDS1 gene.

(32) The transformed plant cells or transgenic plant, or genome-edited plant according to (29), wherein the gene associated with a resistance mechanism is a PAD4 gene.

(33) The transformed plant cells or transgenic plant, or genome-edited plant according to (29), wherein the gene associated with a resistance mechanism is a PAL gene.

(34) The transformed plant cells or transgenic plant, or genome-edited plant according to (29), wherein the gene associated with a resistance mechanism is an ICS gene.

(35) Transformed plant cells or a transgenic plant with overexpression of a gene associated with SA degradation, obtained by the method according to any one of (18) to (28).

(36) The transformed plant cells or transgenic plant according to (35), wherein the gene associated with SA degradation is the SGT gene.

(37) A method for producing a target protein using a plant, wherein the method comprises:
creating transformed plant cells or a transgenic plant, or a genome-edited plant with suppressed or disrupted expression of a gene associated with a resistance mechanism, by the method according to any one of (5) to (17), before step (ii), simultaneously with step (ii) or after step (ii), transferring into the plant cells or plant tissue a fourth nucleic acid construct for expression of a target protein in plants, to create a transgenic plant expressing the nucleic acid construct.

(38) A method for producing a target protein using a plant, wherein the method comprises:
creating transformed plant cells or a transgenic plant with overexpression of a gene associated with SA degradation, by the method according to any one of (18) to (28), and
before step (ii), simultaneously with step (ii) or after step (ii), transferring into the plant cells or plant tissue a fourth nucleic acid construct for expression of a target protein in plants, to create a transgenic plant expressing the nucleic acid construct.

(39) A method for producing a target protein using a plant, wherein the method comprises:
transferring into transformed plant cells or a transgenic plant, or a genome-edited plant, according to any one of (29) to (36), a fourth nucleic acid construct for expression of a target protein in the plant, and
cultivating the nucleic acid construct-transferred transformed plant cells or transgenic plant, to create a transgenic plant expressing the nucleic acid construct.

(40) The method according to any one of (37) to (39), wherein the fourth nucleic acid construct contains a nucleic acid sequence encoding a target protein.

(41) The method according to (40), wherein the fourth nucleic acid construct has an expression promoter sequence that functions in the plant, a nucleic acid sequence encoding the target protein, and an optionally used terminator sequence that functions in the plant, in that order.

(42) The method according to any one of (37) to (41), wherein the transfer of the fourth nucleic acid construct into the plant is carried out using a transient expression system.

(43) Transformed plant cells or a transgenic plant, or a genome-edited plant that produce a target protein, obtained by the method according to any one of (37) to (42).

### Advantageous Effects of Invention

According to the invention, it is possible to create a plant with suppressed resistance to a pathogen by suppression or disruption of expression of a gene associated with a resistance mechanism in plants, or by overexpression of a gene associated with SA degradation in plants. According to the invention, it is also possible to obtain high expression of a target protein in plants.

### BRIEF DESCRIPTION OF DRAWINGS

FIG. 1 is a diagram schematically showing the structure of a plant expression vector (pGPTV-IR-NPR) for the inverted repeat sequence of a partial sequence of an NPR gene (IR-NPR). In the diagram, “pAg7” represents the agropine synthesis gene terminator, “HPT” represents the hygromycin resistance gene, “NOSp” represents the nopaline synthase (NOS) promoter, and “Promoter” represents the strawberry vein binding virus (SVBV)-derived promoter. Also, “NPR” represents the antisense strand and sense strand, in that order, of a partial sequence 400 bp (SEQ ID NO: 1) of the NPR gene. “Terminator” represents the heat shock protein-derived terminator.

FIG. 2 is a diagram schematically showing the structure of an iPAL plant expression vector (pBI-iPAL). In the diagram, “NOSp” represents NOS promoter, “NPTII” represents the kanamycin resistance gene, “NOSt” represents the transcription terminator of the 3′-untranslated region of the NOS gene, and “35Sp” represents cauliflower mosaic virus (CaMV)-derived promoter. “NbPAL” represents the sense strand and antisense strand, in that order, of the partial sequence 157 bp (SEQ ID NO: 6) of the Nicotiana benthamiana (Nb)-derived PAL gene.

FIG. 3 is a diagram schematically showing the structure of an iICS plant expression vector (pBI-iICS). In the diagram, “NOSp” represents NOS promoter, “NPTII” represents the kanamycin resistance gene, “NOSt” represents the transcription terminator of the 3′-untranslated region of the NOS gene, and “35Sp” represents cauliflower mosaic virus (CaMV)-derived promoter. “NbICS” represents the sense strand and antisense strand, in that order, of a partial sequence 153 bp (SEQ ID NO: 9) of the Nb-derived ICS gene.

FIG. 4 is a diagram schematically showing the structure of an SGT plant expression vector (pGPTV-HPT-SGT). In the diagram, “pAg7” represents the agropine synthesis gene terminator, “HPT” represents the hygromycin resistance gene, “NOSp” represents NOS promoter, “Promoter” represents strawberry vein binding virus (SVBV)-derived promoter, “NbSGT” represents an Nb-derived SGT gene, 1368 bp (SEQ ID NO: 12), and “NOSt” represents the transcription terminator at the 3′-untranslated region of the NOS gene.

FIG. 5 is a photograph showing analysis results for Western blotting to determine the expression level of SGT protein accumulating in SGT-transformed Nicotiana benthamiana tobacco obtained by plant tissue culture.

FIG. 6 is a photograph showing results of analysis by Western blotting of a target protein green fluorescent protein (GFP) transiently expressed by agroinfiltration, in order to study expression levels of the target protein (GFP), using NPR-suppressed N. benthamiana (IR-NPR-transformed benthamiana) obtained by plant tissue culture. At the lower end of the diagram are shown target protein (GFP) accumulated amounts (relative to the wild type as 1) and NPR gene mRNA levels (relative to the wild type as 1), in inoculated plants.

FIG. 7 is a photograph showing results of analysis by Western blotting of a target protein (GFP) transiently expressed by agroinfiltration, in order to study expression levels of GFP using PAL-suppressed N. benthamiana (iPAL-transformed N. benthamiana) obtained by plant tissue culture. At the lower end of the diagram are shown target protein (GFP) accumulated amounts (relative to the wild type as 1), PAL gene mRNA levels (relative to the wild type as 1) in inoculated plants, and SA levels (nmol SA/g dry weight) in inoculated individuals.

FIG. 8 is a graph showing RT-PCR analysis results for ICS gene expression levels in ICS-suppressed plants (iICS-transformed N. benthamiana) obtained by plant tissue culture. Defining the expression level of ICS gene in the wild type (WT) plant as 1, individuals of four lines (Nos. 13, 43, 5, 14) were obtained having expression levels lowered to 0.4 or below, among the ICS-suppressed plants.

FIG. 9 is a photograph showing results of analysis by Western blotting of a transiently expressed target protein (GFP), by CMV-agroinfection (vacuum infiltration), in order to study expression levels of GFP using SGT-transformed N. benthamiana (overexpressing plant) obtained by plant tissue culture. Expression of the target protein (GFP) in inoculated plants was confirmed only in the leaf veins in the wild type plants, but was confirmed in both the leaf veins and mesophyll in the SGT-overexpressing plants.

FIG. 10 is a photograph showing GFP fluorescence in the leaves of SGT-transformed tobacco (SGT-overexpressing plant, two top photographs) and wild type N. benthamiana (wild type plant, two bottom photographs), of FIG. 9. The light areas in the photographs (green in the originals) indicate the GFP expression sites, and the dark areas (black in the originals) indicate the sites where GFP luminescence could not be clearly confirmed.

FIG. 11 is a graph showing measurement results for amounts of salicylic acid (SA) and SA metabolites (SAG, SGE) accumulated in the SGT-overexpressing plant and wild type plant shown in FIG. 10. In the (Agrobacterium) non-inoculated individual, almost no SA or SA metabolites were detected, whereas in the individual inoculated with Agrobacterium harboring CMV-agroinfection vector-SA and SA metabolites were detected, with more accumulation of SA metabolites being detected in the SGT overexpressing plant than the wild type plant.

FIG. 12 is a diagram schematically showing the structure of a plant expression vector (pEgP237-2A-GFP-NPRgRNA) for Cas9 and guide RNA expressions (Ueta R et al., Sci Rep 7:507, 2017). In the diagram, “AtU6-26” represents AtU6-26 promoter, “gRNA” represents guide RNA, “35SpΩ” represents the sequence of cauliflower mosaic virus-derived 35S promoter with the Ω sequence attached as a translation enhancer, “CAS9” represents Cas9, “NLS” represents the nuclear localization signal, “2A” represents 2A auto-cleaving peptide, “Terminator” represents the Arabidopsis thaliana-derived terminator, and “Km^{r}” represents the kanamycin resistance gene. “NPRg526”, “NPRg524” and “NPRg417” represent specific guide RNAs, having the nucleotide sequences of SEQ ID NOs: 22 to 24, respectively.

FIG. 13 is a diagram schematically showing the structure of a plant expression vector (pEgP237-2A-GFP-NPRgRNA) for Cas9 and guide RNA expressions. In the diagram, “AtU6-26” represents AtU6-26 promoter, “gRNA” represents guide RNA, “35SpΩ” represents the sequence of cauliflower mosaic virus-derived 35S promoter with the Ω sequence attached as a translation enhancer, “CAS9” represents Cas9, “NLS” represents the nuclear localization signal, “2A” represents 2A auto-cleaving peptide, “Terminator” represents the Arabidopsis thaliana-derived terminator, and “Km^{r}” represents the kanamycin resistance gene. “ICSg362”, “ICSg121” and “ICSg95” represent specific guide RNAs, having the nucleotide sequences of SEQ ID NOs: 25 to 27, respectively.

FIG. 14 is a photograph showing results of analysis by Western blotting of an antibody (IgG) transiently expressed by agroinfiltration, in order to study expression levels of the target protein (IgG), using NPR genome-edited N. benthamiana (NPRg526 no. 72-3) obtained by plant tissue culture. At the lower end of the diagram are shown accumulated amounts (relative to the wild type as 1) of the target protein (IgG) in inoculated plants.

FIG. 15 is a photograph showing results of analysis by Western blotting of IgG transiently expressed by agroinfiltration, in order to study expression levels of the target protein (IgG), using ICS genome-edited N. benthamiana obtained by plant tissue culture. At the lower end of the diagram are shown target protein (IgG) accumulated amounts (relative to the wild type as 1) in inoculated plants, and SA levels (nmol SA/g dry weight) in inoculated individuals.

FIG. 16 is a schematic diagram showing a time series for an example of expressing a target protein (GFP) after obtaining EDS1 expression-suppressed N. benthamiana and PAD4 expression-suppressed N. benthamiana by the VIGS method. The photographs show GFP fluorescence exhibited in the plants. The light areas in the photographs (green in the originals) indicate the GFP expression sites, and the dark areas (black in the originals) indicate the sites where GFP luminescence could not be clearly confirmed. The numbers under “Examining gene suppression by quantitative RT-PCR” represent relative values for EDS gene expression level in EDS1 expression-suppressed N. benthamiana or relative values for PAD4 gene expression level in PAD4 expression-suppressed N. benthamiana, where expression level of the EDS1 gene or PAD4 gene in the wild type plant (no gene expression suppression) was defined as 1.

FIG. 17 is a photograph showing results of analysis by Western blotting of a target protein (GFP) transiently expressed by agroinfiltration, in order to study expression levels of GFP using the EDS1 expression-suppressed N. benthamiana and PAD4 expression-suppressed N. benthamiana shown in FIG. 16. At the lower end of the diagram are shown target protein (GFP) accumulated amounts (relative to the wild type as 1) in inoculated plants, and SA levels (nmol SA/g dry weight) in inoculated individuals.

FIG. 18 is a diagram schematically showing the structure of a plant expression vector (pEgP237-2A-GFP-EDSgRNA) for Cas9 and guide RNA expressions. In the diagram, “AtU6-26” represents AtU6-26 promoter, “gRNA” represents guide RNA, “35SpΩ” represents the sequence of cauliflower mosaic virus-derived 35S promoter with the Ω sequence attached as a translation enhancer, “CAS9” represents Cas9, “NLS” represents the nuclear localization signal, “2A” represents 2A auto-cleaving peptide, “Terminator” represents the Arabidopsis thaliana-derived terminator, and “Km^{r}” represents the kanamycin resistance gene. “EDSg715”, “EDSg533”, “EDSg429” and “EDSg322” represent specific guide RNAs, having the nucleotide sequences of SEQ ID NO: 47 to 50, respectively.

FIG. 19 is a photograph showing results of analysis by Western blotting of a target protein (GFP) transiently expressed by agroinfiltration, in order to study expression levels of GFP using SGT-transformed N. benthamiana obtained by plant tissue culture. At the lower end of the diagram are shown accumulated amounts (relative to the wild type as 1) of the target protein (GFP) in inoculated plants.

FIG. 20 is a photograph showing results of analysis by Western blotting of an antibody (IgG) transiently expressed by agroinfiltration, in order to study expression levels of the target protein (IgG), using NPR genome-edited N. benthamiana obtained by plant tissue culture. Used were the genetically fixed plant in progeny of NPR genome-edited N. benthamiana (NPRg526 no. 72-3-40) (the plant expression vector of FIG. 12 was separated by a genetic method and is not included) shown in FIG. 14, and a plant (NPRg524 no. 567) obtained by genome editing using different guide RNA (NPRg524). At the lower end of the diagram are shown accumulated amounts (relative to the wild type as 1) of the target protein (IgG) in inoculated plants.

FIG. 21 is a photograph showing results of analysis by Western blotting of an antibody (IgG) transiently expressed by agroinfiltration, in order to study expression levels of the target protein (IgG), using NPR genome-edited N. benthamiana obtained by plant tissue culture. Used here was a plant (NPRg524 no. 444) obtained by genome editing using guide RNA (NPRg524). At the lower end of the diagram are shown accumulated amounts (relative to the wild type as 1) of the target protein (IgG) in inoculated plants.

### DESCRIPTION OF EMBODIMENTS

The invention will now be described in greater detail by concrete embodiments thereof. However, it is to be understood that the invention is not restricted in any way to the following embodiments, and various modifications thereof may be implemented.

### First Embodiment

The first embodiment of the invention relates to a method for creating a plant with suppressed resistance to a pathogen, wherein the method comprises suppressing or disrupting expression of a gene associated with a resistance mechanism in the plant.

The term “resistance mechanism in a plant” refers to a self-defense mechanism possessed by the plant against a variety of different external stresses (Ethan et al., Disease Resistance Mechanisms in Plants, Genes 2018, 9(7), 339). For example, plants synthesize abscisic acid (ABA) when detecting changes in the environment (such as dryness, low temperature or high salt concentration), salicylic acid (SA) in response to infection by pathogens, and jasmonic acid (JA) in response to feeding damage by insects, and these low molecular compounds act as signal transducers to activate a response system against such stresses and thus protect the plant.

The term “pathogen” includes filamentous fungi (mold and fungi), bacteria, viruses, viroids, phytoplasma, Rickettsiosis microorganisms, nematodes, protozoans, etc. About 80% or more of infectious diseases are due to filamentous fungi.

The “gene associated with a resistance mechanism” is not particularly restricted so long as it is a gene associated with a resistance mechanism, and for example, it may be a gene associated with the SA biosynthetic pathway, a gene associated with SA signal transduction, or a gene associated with SA degradation, as described for the second embodiment.

As used herein, “SA biosynthetic pathway” refers to a pathway leading to biosynthesis of SA, while “SA biosynthesis” refers to metabolism of a SA precursor. The term “SA precursor” may refer to chorismic acid, isochorismic acid, L-phenylalanine, trans-cinnamic acid, cinnamoyl-CoA, benzoyl-CoA, ortho-coumaric acid, benzoic acid, etc. (Ishihama, N., Shirasu, K., Regulation of Plant Growth & Development Vol. 53, No. 1, 53-59, 2018). The term “SA degradation” refers to degradation or metabolism of already biosynthesized SA.

Pathways of SA biosynthesis, degradation and signal transduction are well-known (for example, Weijie Huang et al., Molecular Plant, 13, 31-41, January 2020; Muhammad Saad Shoaib Khan et al., Frontiers in Plant Science, January 2020, Volume 13, 1-12|Article 82937; Pingtao Ding et al., Trends in Plant Science, June 2020, Vol. 25, No. 6, 549-565), and may be summarized as follows.

SA is biosynthesized by two pathways, the isochorismate synthase (ICS) pathway and the phenylalanine ammonia-lyase (PAL)-mediated pathway. Chorismic acid (CA) is converted to isochorismic acid (IC) by ICS in plastids. The IC is transported by MATE (multidrug and toxin extrusion protein) transporter EDS5 (enhanced disease susceptibility 5), to enter the cytoplasm and be converted to isochorismoyl-9-glutamate (IC-9-Glu) by PBS3 (avrPphB Susceptible 3). IC-9-Glu is then spontaneously cleaved to become SA. EPS1 (enhanced pseudomonas susceptibility 1) enhances this cleavage. In the PAL-mediated pathway, phenylalanine is converted to trans CA by PAL, the trans CA being in turn converted by AIM1 (abnormal inflorescence meristem 1) to benzoic acid via beta-oxidation, and the benzoic acid then being converted to SA (Weijie Huang et al., Molecular Plant, 13, 31-41, January 2020).

NPR1 (nonexpressor of pathogenesis-related genes 1) is present as an important signal transduction factor downstream from SA, and after receiving the SA signal it is reduced and separated into monomers, after which it migrates to the nucleus where it acts with coactivators such as EDS1 (enhanced disease susceptibility 1) and cyclin-dependent kinase 8 (CDK8) to induce expression of PR (pathogenesis related) genes (Muhammad Saad Shoaib Khan et al., Frontiers in Plant Science, January 2020, Volume 13, 1-12|Article 82937; Pingtao Ding et al., Trends in Plant Science, June 2020, Vol. 25, No. 6, 549-565). SA regulates transcription activity of NPR1 via NPR3/4. In addition, downstream from NPR1, several plant-specific WRKY transcription factors are induced in a SA-dependent manner, which are believed to be involved in expression of defensive response genes.

Phytoalexin Deficient 4 (PAD4) encodes a lipase-like gene and is thought to interact with EDS1 to control the defensive response. SA is also glycosylated by salicylic acid glucosyltransferase (SGT), etc. The glycoside is a storage body, without itself exhibiting activity, but it may be hydrolyzed, if necessary, to form free SA.

Examples of genes associated with the SA biosynthetic pathway include the phenylalanine ammonia-lyase (PAL) gene, isochorismate synthase (ICS) gene, EDS1 gene, EDS5 gene, Phytoalexin Deficient 4 (PAD4) gene, salicylate decarboxylase (SDC) gene, salicylic acid glucoside hydrolase gene, AIM1 (abnormal inflorescence meristem 1) gene, PBS3 (avrPphB susceptible 3) gene, EPS1 gene, SARD1 (SAR-deficient 1) gene and calmodulin binding protein 60g (CBP60g) gene.

The gene associated with SA signal transduction may be an NPR gene (nonexpressor of pathogenesis-related genes), for example, the NPR gene being specifically a gene such as the NPR1 gene or NPR3/4 gene.

As used herein, “suppresses expression of a gene” refers to causing no production or reduced production volume of a protein encoded by a gene associated with a resistance mechanism in the plant of interest (hereafter also referred to as “target gene” as appropriate), and the suppressed expression includes the temporarily suppressed expression. Suppression may be inhibition of the transcription step of the target gene from DNA to mRNA, or inhibition of the translation step of the gene from mRNA to protein, or decomposition of the mRNA. The degree of inhibition is not particularly restricted and may be enough to suppress resistance to a pathogen in the plant having suppressed expression of the gene.

As used herein, “disrupt expression of a gene” means that the target gene does not function in the plant of interest and therefore the protein having the normal function encoded by the gene is not produced.

The gene associated with SA degradation will now be explained using the second embodiment.

For the first embodiment of the invention, the method of the invention may comprise:
(i) a step of preparing a nucleic acid construct for suppression or disruption of the gene associated with a resistance mechanism,
(ii) a step of transferring the nucleic acid construct into plant cells or plant tissue, and
(iii) a step of culturing the plant cells or plant tissue having the transferred nucleic acid construct to create a plant expressing the nucleic acid construct. This method will be referred to as “Embodiment 1-1”, as appropriate.

### [First Nucleic Acid Construct]

The nucleic acid construct of step (i) preferably contains a nucleic acid sequence complementary to the mRNA of the target gene or transcriptional product thereof (also referred to as “first nucleic acid construct” or “nucleic acid construct containing a nucleic acid sequence complementary to target gene mRNA, etc.”). In the first nucleic acid construct, the nucleic acid sequence complementary to the mRNA of the target gene or transcriptional product thereof may be antisense RNA, an RNA interference (RNAi) molecule or a virus-induced gene silencing (VIGS) molecule, for example.

In step (i), the nucleic acid construct may be created in the following manner.

Firstly, the method for suppression or disruption of gene expression is as follows.

The method for suppression or disruption of gene expression is not particularly restricted and may be appropriately selected depending on the purpose.

The method for suppression of gene expression may be, for example, an RNA interference (RNAi) method, an antisense method, or a temporary gene suppression method such as virus-induced gene silencing (VIGS). It may also be a ribozyme method in which the target gene transcriptional product is cleaved, or a co-inhibition method in which a co-inhibition effect during protein expression is used to suppress expression of the gene encoding the protein.

The method for disrupting expression of the gene may be genome editing, for example.

When an RNAi method is used, the method may be transfer of DNA coding for RNA that suppresses the target gene expression by an RNAi effect during target gene expression using an RNAi vector, for example. This method suppresses target gene expression by RNA interference (RNAi) with double stranded RNA having the same or a similar sequence as the nucleotide sequence of the gene.

The RNAi vector is preferably a vector that expresses RNAi-eliciting dsRNA, as hairpin dsRNA. The dsRNA-expressing RNAi vector that is used may be a hairpin RNAi vector constructed by inserting DNA corresponding to the dsRNA-forming portion, so that it forms an IR (inverted repeat) at both ends of a spacer sequence of at least several bases, such as an intron.

The RNAi vector may be a tandem type that is transcribed to sense RNA and antisense RNA respectively, by a promoter, which hybridize in the cells to produce dsRNA. RNAi may also be induced by constructing several expression vectors designed so that sense RNA and antisense RNA are both transcribed.

According to the invention, “RNA interference (RNAi) molecule” is used to refer to a sequence of double stranded RNA that induces RNAi.

The method for suppression of gene expression is preferably suppress of gene expression by DNA methylation (transcriptional gene silencing). RNAi also participates in regulation of gene expression at the transcription level by DNA methylation or chromatin modification, and in plants it is known that methyl group addition reaction takes place at the 5-position carbon atom of the pyrimidine ring of cytosine.

When using an antisense method, the method may be introduction of DNA coding for antisense RNA complementary to at least part of the transcriptional product of the target gene. This method suppresses target gene expression in a plant by transfer of DNA coding for antisense RNA complementary to at least a portion of the transcriptional product of the gene.

One function of antisense RNA as suppression of target gene expression is inhibition of transcription initiation by formation of triplexes, thereby suppressing the target gene expression via inhibition of the different processes such as transcription, splicing and translation. According to the invention, the target gene expression may be suppressed by any of the aforementioned functions.

The nucleotide sequence of the antisense RNA is preferably a sequence complementary to at least a portion of the transcriptional product of the target gene, and it does not need to be completely complementary so long as it can effectively suppress the target gene expression.

For example, the sequence identity between the DNA strand complementary to DNA coding for the antisense RNA, and the target gene, is preferably at least 80%, at least 85%, at least 90%, at least 91%, at least 92%, at least 93%, at least 94%, at least 95%, at least 96%, at least 97%, at least 98% or at least 99%. The sequence identity referred to here is the value that can be calculated by appropriately aligning at least two sequences to be compared, determining the identical residues in each of the sequences, determining the number of matching sites, and then dividing the number of matching sites by the total number of residues in the sequence region to be compared, and multiplying the resulting value by 100. Specifically, the sequence identity can be calculated using the commonly used BLAST algorithm at http://www.ncbi.nlm.nih.gov/BLAST/, for example. The length of the DNA coding for the antisense RNA may be appropriately set as desired, without any particular restrictions.

Virus-induced gene silencing (VIGS) is an expression suppression technique for coding genes in plants utilizing the RNA silencing mechanism induced by plant virus infection. For example, if part of the sequence of a target gene in a plant in which it is desired to suppress expression is transferred into a cloning site of a plasmid having transferred viral genome DNA (a vectorized plasmid), and the plasmid is used to infect the plant with the viral vector, then plant mRNA homologous to the transferred plant gene sequence is degraded, thus resulting in specific knockdown of its expression. For targeting of multiple genes, the sequence of the siRNA is designed by selecting a region within the coding regions of homologous genes where the 21nt siRNA completely matching the sequence is maximally conserved. The advantage of a VIGS system is that it does not require a plant transformation protocol matching the plant variety in which the target gene is to be silenced. As used herein, “VIGS molecule” refers to a partial sequence of the target gene in the plant whose expression is to be suppressed.

In VIGS it is not possible to completely suppress target gene expression (see Purkayastha A. et. al., Plant Physiology and Biochemistry vol. 47, 967-976, 2009, for example). Specifically, the phenotype in VIGS is affected by the type (function) or degree of suppression of the target gene, and the range within which VIGS is induced. Depending on the vector and plant variety, therefore, it may only be possible to induce partial silencing, or depending on the viral vector, the inserted gene may be unstable and be shed within a short period.

The method for expressing a nucleic acid sequence complementary to the mRNA of the target gene or transcriptional product thereof in a plant is not particularly restricted, and any method publicly known to a person skilled in the art may be used as appropriate. An example is a method in which an expression cassette having a complementary nucleic acid sequence linked in the antisense direction downstream from the promoter is constructed, and the expression cassette is inserted into plant cells. The method of constructing the expression cassette may be selected as appropriate depending on the purpose. For example, a first nucleic acid construct can be constructed by operationally linking a promoter sequence that can be transcribed in plants, the aforementioned complementary nucleic acid sequence, and optionally an appropriate terminator sequence, in that order.

Introduction of the first nucleic acid construct into a plant in step (ii) may be carried out using a stable expression system or a transient expression system.

In a transient expression system, when a gene encoding the target protein introduced into host cells (hereafter referred to as “exogenous gene” as appropriate) is expressed, the exogenous gene is expressed separately from the chromosomes of the host cells. It is therefore possible to express the exogenous gene in about 3 days to about 2 weeks, which is effective for methods of producing target proteins in plants. Expression of the exogenous gene is attenuated and disappears as time passes. In a stable expression system, on the other hand, the exogenous gene is incorporated into the chromosomes, allowing stable expression over long periods.

Methods for transferring exogenous genes into plants in a stable expression system are largely classified as either direct methods such as PEG, electroporation and particle gun methods, and Agrobacterium methods.

In an Agrobacterium method, a T-DNA (transferred-DNA) region in a Ti plasmid which is conserved in Agrobacterium is incorporated into the plant chromosomes by the action of a gene group associated with DNA transfer in the same Ti plasmid, and another gene group present in Agrobacterium or plant chromosomes. The plant cells into which the T-DNA has been incorporated produce plant hormones, resulting in formation of crown gall. The Agrobacterium method utilizes the ability of Agrobacterium to incorporate its own DNA into plant cells, replacing the portion flanked by the left and right boundary regions (LB and RB) of the T-DNA region with the exogenous gene, thus allowing introduction of the exogenous gene into the plant. In the most common Agrobacterium method, first, a binary vector is constructed having an exogenous gene incorporated in the T-DNA region by genetic engineering using E. coli, and the vector is introduced into Agrobacterium. The recombinant Agrobacterium with the binary vector is cultured by liquid culture, and the cultured cells are contacted with plant sections to establish infection. A plant hormone is then added to the medium to induce regeneration of the plant cells, and plant cells with chromosomal insertion of the T-DNA region containing a selective marker such as antibiotic resistance are screened based on antibiotic resistance, etc., in a callus state, to create a regeneration recombinant plant.

The transient expression system used may be an agroinfiltration method or plant viral vector method, or an agroinfection method that combines both. A transient expression system does not include a regeneration step as described above for a stable expression system.

In a plant viral vector method, cDNA of a plant virus genome with the exogenous gene inserted is transcribed in vitro, the obtained RNA is inoculated as vector into a plant for infection, and the proliferation potency and systemic transferability/movement of the virus itself are used for expression of the exogenous gene in the plant. An example of a plant viral vector is selected from the group consisting of full viral vectors, and deconstructed viral vectors such as tobacco mosaic virus (TMV), plum pox virus (PPV), turnip vein-clearing virus (TVCV), potato virus X (PVX), bean yellow dwarf virus (BEYDV), alfalfa mosaic virus (AIMV), cucumber mosaic virus (CMV), cowpea mosaic virus (CPMV), zucchini yellow mosaic virus (ZYMV), tobacco rattle virus (TRV), apple latent spherical virus (ACMV), bromo mosaic virus (BMV), tomato mosaic virus (ToMV), tomato yellow leaf curl virus (TYLCV) and tomato golden mosaic virus (TGMV).

In the agroinfection method, a replicon that is able to self-propagate/replication in plant cells is inserted into the T-DNA region to induce Agrobacterium infection, after which the exogenous gene is self-propagated/replication by its own proliferation power. The agroinfection method allows expression of an exogenous gene in tissues throughout the plant, and can therefore increase expression levels of the exogenous gene per plant cell. An agroinfection method previously developed by the present inventors which combines the CMV and T-DNA sequence (International Patent Publication No. 2016/0002654) is able to express an exogenous gene throughout a plant and at a high level in virtually all cells of the plant.

In case of having the first nucleic acid construct expressed in a plant using, for example, a stable expression system in an Agrobacterium-mediated transformation method or Agrobacterium has the following sequences (a) to (e) in order. Sequence (d), however, is optional.
(a) Right border sequence (RB) derived from the Agrobacterium T-DNA sequence;
(b) expression promoter sequence that functions in the plant;
(c) nucleic acid sequence complementary to mRNA of the target gene or a transcriptional product thereof;
(d) terminator sequence that functions in the plant;
(e) left border sequence (LB) derived from the Agrobacterium T-DNA sequence.

Sequences (a) and (e), i.e. the right border sequence (RB) and left border sequence (LB) derived from the Agrobacterium T-DNA sequence, are as described above.

The type of expression promoter sequence of (b) is not particularly restricted so long as it is a sequence that can function in the plant genome and start transcription of the nucleic acid sequence of (c). Examples of such promoters include Agrobacterium-harboring NOS promoter, cauliflower mosaic virus (CaMV) 35S promoter (Odell et al., (1985), Nature, 313:810-812), cassava mosaic virus promoter, figwort mosaic virus promoter, Badnavirus promoter, Strawberry vein binding virus (SVBV) promoter, Mirabilis mosaic virus promoter (MMV), Rubisco promoter, actin promoter, ubiquitin promoter, etc. The cauliflower mosaic virus (CaMV) 35S-derived promoter is preferred among these.

The terminator sequence of (d) is an optional element, but a terminator sequence is preferably linked from the viewpoint of reliably stopping transcription of the nucleic acid sequence of (c) and expressing the desired functional protein. The type of terminator sequence is not particularly restricted so long as it is a sequence that can stop transcription of the coding sequence of the nucleic acid sequence of (c). Examples include Agrobacterium-harboring NOS terminator, heat shock protein (hsp) terminator and cauliflower mosaic virus (CaMV) 35S terminator, etc.

The first nucleic acid construct may further include other sequences so long as its function is not substantially impeded. Examples of such other sequences include any other sequence derived from Agrobacterium Ti plasmid (such as the vir region), and selective marker genes, etc.

A selective marker gene is used to confirm transfer of the nucleic acid construct. Its type is not particularly restricted, but it will usually be an antibiotic resistance gene or drug resistance gene, such as a gene conferring resistance to ampicillin, streptomycin, gentamicin, kanamycin, hygromycin, actinonin (PDF1 gene), bialaphos herbicides, glyphosate herbicides, sulfonamide, mannose, etc. The selective marker gene is usually operationally linked to a regulatory sequence such as a unique promoter, and constructed as an expression cassette that is autonomously expressed in the plant genome, while being situated between the right border sequence (RB) and left border sequence (LB). Randomly incorporating the right border sequence (RB) and left border sequence (LB) into the genome results in introduction of the selective marker into the plant genome, and its autonomous expression.

As mentioned above, the first nucleic acid construct can also be expressed by agroinfection, and it is especially preferred to use an agroinfection method combining the CMV vector and T-DNA sequence (International Patent Publication No. 2016/0002654). In this case, the first nucleic acid construct will have (a) to (e) in the following order. Sequence (d), however, is optional.
(a) right border sequence (RB) derived from Agrobacterium T-DNA sequence;
(b) expression promoter sequence that functions in the plant;
(c′) sequence corresponding to the CMV RNA2 genome, wherein all or a portion of the gene encoding the 2b protein is replaced with a nucleic acid sequence complementary to mRNA of the target gene or its transcriptional product thereof,
(d) terminator sequence that functions in the plant; and
(e) left border sequence (LB) derived from Agrobacterium T-DNA sequence.

In the sequence of (c′), the sequence corresponding to the RNA2 genome of CMV will usually be the cDNA sequence of the RNA2 genome. The CMV RNA2 genome includes the gene encoding the 2a protein and the gene encoding the 2b protein. According to the invention, all or a portion of the sequence corresponding to the gene encoding the 2b protein in the cDNA sequence of the RNA2 genome is replaced with a nucleic acid sequence complementary to the mRNA of the target gene or transcriptional product thereof.

The first nucleic acid construct may be in a linear or circular form. However, a circular form is preferably in the form of a plasmid, for example, and especially in the form of a T-DNA vector having the ability to replicate in Agrobacterium.

A nucleic acid construct having such a structure can be easily created using appropriate combinations of gene recombinant techniques well-known to a person skilled in the art.

When a stable expression system in Agrobacterium is used, the method for transferring the first nucleic acid construct into a plant in step (ii) of Embodiment 1-1 or 1-2 is, preferably, for example, preparation of a solution containing the first nucleic acid construct (cultured cells), and transfer of the solution into plant tissue by various physical methods such as dipping or bacterial coating.

When a transient expression system is used, the method for transferring the first nucleic acid construct into a plant in step (ii) of Embodiment 1-1 is, preferably, for example, preparation of a solution containing the first nucleic acid construct, and transfer of the solution into plant tissue by, for example, infusion or permeation.

Specifically, the solution containing the first nucleic acid construct may be a cultured product obtained by culturing the transformed Agrobacterium (such as an overnight cultured product). An overnight cultured product generally reaches an optical density (OD600) of 3 to 3.5 units at a wavelength of 600 nm. This is usually diluted 3 to 5 times before agroinfiltration to produce 5 to 9×10⁹ colony forming units (Turpen et al., (1993), J. Virol. Methods, 42:227-240). The cultured product is suspended in MES buffer, for example, to OD600=0.2 to 0.8. When each bacterial cell solution is to be used alone, it is prepared to a suspension with OD600=0.2 to 0.6. When several bacterial cell solutions are to be used in combination, they are mixed in equal cellular amounts for preparation to a final suspended bacterial amount of OD600=0.3 to 1.2.

When a solution containing the first nucleic acid construct is to be introduced into a plant by infusion, a syringe, for example, may be used to forcibly inject the solution into the plant. When a solution containing the nucleic acid construct is to be introduced into a plant by permeation, the solution containing the nucleic acid construct is contacted with the plant, and a vacuum desiccator, for example, is then used to create reduced pressure conditions (about −0.09 Mpa), for forcible permeation of the solution into the plant.

The plant used in the method of the invention is not particularly restricted and may be any arbitrary species of plant. It may be the grown plant, plant cells, plant tissue, callus or seeds, for example. Plant cells include a variety of different plant cell types. Examples of such plant cells include suspension culture cells, protoplasts and leaf slices.

Examples of plants to which the invention may be applied include alfalfa, barley, green bean, canola, cowpea, cotton, corn, clover, lotus, lentil, lupin, millet, oat, pea, peanut, rice, rye, sweet clover, sunflower, sweet pea, soybean, sorghum, triticale, jicama, velvet bean, horse-bean, wheat, wisteria and nut plants.

Preferable plants include plants belonging to Poaceae, Compositae, Solanaceae and Rosaceae.

Plants of the following types are even more preferred. Thale cress, redtop grass, Welsh onion, snapdragon, dutch celery, peanut, asparagus, Atropa, wild oat, thorny bamboo, rape, bromegrass, rurimagaribana, camellia, cannabis, capsicum, chickpea, goosefoot, chicory, citrus, coffee tree, juzudama, cucumber, pumpkin, bermudagrass, dactylis, jimsonweed, gourd, digitalis, yam, oil palm, ooshiba, fescue, strawberry, geranium, soybean, sunflower, Hemerocallidoideae, Para rubber plant, barley, henbane, sweet potato, lettuce, Lens culinaris, lily, flax, ryegrass, lotus, tomato, marjoram, apple, mango, manihot, burr medic, African toadflax, tobacco, onobrychis, rice, millet, geranium, Chinese fountain grass, petunia, pea, kidney bean, phleum, meadow grass, cherry flower, buttercup, radish, currant, castor bean, raspberry, sugarcane, Salpiglossis, rye, senecio, setaria, white mustard, eggplant, sorghum, buffalo grass, cacao, clover, Trigonella caerulea, wheat, horse-bean, cowpea, grape and corn, etc.

In step (iii), the plant having the first nucleic acid construct introduced is cultivated to express the nucleic acid sequence complementary to the mRNA of the target gene or transcriptional product thereof, which has been incorporated into the nucleic acid construct. The method of cultivating the plant may be selected as appropriate depending on the type of plant and the purpose of expressing the complementary nucleic acid sequence.

According to the first embodiment of the invention, the method of the invention may comprise:
(i) a step of preparing a second nucleic acid construct containing a nucleic acid sequence encoding a genome editing-related protein with a target site in the gene associated with a resistance mechanism,
(ii) a step of transferring the nucleic acid construct into plant cells or plant tissue, and
(iii) a step of culturing the plant cells or plant tissue having the transferred nucleic acid construct to create a plant with a mutation transferred into the gene sequence. This method will be referred to as “Embodiment 1-2”, as appropriate.

Embodiment 1-2 may further comprises, after step (iii), a step (iv) in which the second nucleic acid construct is removed from the plant with a mutation transferred into the gene sequence. For example, seeds that inherit the nucleotide sequence altered by genome editing but without the second nucleic acid construct may be selected from seeds harvested by growing the successfully genome edited plant (i.e., the next generation from genome editing).

### [Second Nucleic Acid Construct]

The nucleic acid construct of step (i) may also contain, within the target gene, a nucleic acid sequence encoding a genome editing-related protein with a target site (“second nucleic acid construct,” or “nucleic acid construct for expression of genome editing-related protein”). The genome editing-related protein used may be CRISPR-Cas9 (Clustered Regularly Interspaced Short Palindromic Repeats CRISPR-Associated Proteins 9), TALEN (Transcription Activator Like Effector Nuclease), ZFN (Zinc Finger Nuclease), PPR (Pentatricopeptide Repeat), etc. In order to impart site specificity to the genome editing-related protein, a sequence recognition domain that binds to the target gene is used with ZFN and TALEN (ZF domain, TALE domain), while a guide RNA having a sequence complementary to the target gene is used with CRISPR/Cas9. A CRISPR-Cas9 system, in particular, has a simpler structure, a high degree of sequence design freedom, and a satisfactory genome editing success rate, as well as low cost.

Several Cas9 proteins are known (see U.S. Pat. Nos. 8,697,359, 8,865,406 and International Patent Publication No. 2013/176772, for example), and may be used. In a known CRISPR/Cas system, it is common to use Cas9 derived from Streptococcus pyogenes. Cas9 protein can be obtained by expression and recovery from a publicly known Cas9 expression vector using appropriate host cultured cells. The Cas9 protein used may be a commercial product.

The Cas9 protein may further include a molecule or molecular complex involved in specific recognition of the target gene. Examples include nucleic acid sequence-recognition modules and guide RNA.

The term “nucleic acid sequence-recognition module” refers to a molecule or molecular complex having the ability to specifically recognize and bind to a target gene, and for example, it may be a protospacer adjacent motif (PAM) sequence, or DNA binding domain of a protein that can specifically bind to DNA, such as a restriction enzyme, transcription factor or RNA polymerase. The PAM sequence will vary depending on the species or type of the Cas9 protein used.

Guide RNAs guide Cas9 protein to the target site. Methods for designing and creating guide RNAs are known. Guide RNAs may be a single-molecule guide RNA (sgRNA) such as crRNA (CRISPR RNA) or tracrRNA (trans-activating crRNA) or a double-molecule guide RNA comprising a crRNA fragment and tracrRNA fragment. The targeted sequence in the crRNA will usually consists of 12 to 50 nucleotides, preferably 17 to 30 nucleotides and more preferably 17 to 25 nucleotides, and is selected so as to target a microinjection region adjacent to the PAM sequence.

The guide RNA may be in the form of RNA, in the form of DNA coding for the RNA, or in the form of a vector expressing the DNA. When an RNA form is used, it can be prepared by chemical synthesis based on the nucleotide sequence, using a commercial polynucleotide synthesizer. It can also be prepared using an in vitro transcription system. According to the invention, the guide RNAs of SEQ ID NOs: 22 to 23 (the PAM sequence in each guide RNA sequence being the 3 nucleotides at the 3′-end) may be used when the purpose is to disrupt the NPR1 gene. When the purpose is to disrupt the ICS gene, the guide RNAs of SEQ ID NOs: 25 to 27 may be used (the PAM sequence in each guide RNA sequence of SEQ ID NOs: 25 and 27 being the 3 nucleotides at the 5′-end, and the PAM sequence in the guide RNA sequence of SEQ ID NO: 26 being the 3 nucleotides at the 5′-end). When the purpose is to disrupt the EDS1 gene, the guide RNAs of SEQ ID NOs: 47 to 50 (the PAM sequence in each guide RNA sequence of SEQ ID NOs: 47 to 50 being the 3 nucleotides at the 3′-end) may be used.

For TALEN, refer to Japanese Patent No. 8470973, U.S. Pat. No. 8,586,363, and Zhang, Feng et al. (2011) Nature Biotechnology 29(2), and for ZFN, refer to U.S. Pat. No. 6,265,196, No. 8524500 and No. 7888121, and European Patent No. 1720995, for example.

The method for expressing a nucleic acid sequence encoding a genome editing-related protein in a plant is not particularly restricted and may be a method publicly known to a person skilled in the art, as appropriate. For example, the method may be one in which at least part of the target gene is linked with a nucleic acid molecule having a nucleic acid sequence encoding a genome editing-related protein, downstream from a promoter, to construct an expression cassette, and introducing the expression cassette into plant cells. The method of constructing the expression cassette may be selected as appropriate depending on the purpose. For example, a second nucleic acid construct can be constructed by operationally linking a promoter sequence that can be transcribed in plants, a nucleic acid molecule having a nucleic acid sequence encoding a genome editing-related protein, and optionally an appropriate terminator sequence, in that order.

The second nucleic acid construct can be expressed in a plant using a stable expression system, and has the following sequences (a) to (e) in order. Sequence (d), however, is optional.
(a) Right border sequence (RB) derived from the Agrobacterium T-DNA sequence;
(b) expression promoter sequence that functions in the plant;
(c) nucleic acid sequence encoding a genome editing-related protein;
(d) terminator sequence that functions in the plant;
(e) left border sequence (LB) derived from the Agrobacterium T-DNA sequence.

Step (ii) and step (iii) in Embodiment 1-2 are the same as explained for Embodiment 1-1.

### Second Embodiment

The second embodiment of the invention relates to a method for creating a plant with suppressed resistance to a pathogen, wherein the method comprises overexpressing a gene associated with SA degradation in plants (hereafter referred to as “SA degradation-associated gene” or “target gene”, as appropriate).

The phrase “overexpressing a gene” means increasing the production volume of a protein encoded by a target gene in a plant of interest, and it includes increasing an endogenous target gene expression level even without external transfection of the target gene. The level of increase is also not particularly restricted.

Any known method may be used for external introduction of a target gene. The specific method is not restricted, and for example, a recombinant vector may be introduced into the plant to cause expression of the target gene. The target gene expression method may be in a stable expression system or a transient expression system. For increasing an endogenous target gene expression level, a mutant may be acquired having the endogenous target gene expression level increased using a known mutation source.

SA is normally converted by an SA degradation-associated gene to inactive glycosides such as O-glucose salicylate or salicylic acid glucose esters, but overexpression of an SA degradation-associated gene inhibits accumulation of SA and increases production of inactive glycosides. Examples of SA degradation-associated genes include the SA glucosyltransferase (SGT) gene, SA glycosyltransferase genes (UGT74F1, UGT74F2, UGT76B1, UGT75B1), the salicylic acid 3-hydroxylase (S3H) gene, salicylic acid 5-hydroxylase (SSH) gene and salicylate hydroxylase (nahG) gene.

For the second embodiment of the invention, the method of the invention may comprise:
(i) a step of preparing a nucleic acid construct for overexpression of a gene associated with SA degradation,
(ii) a step of transferring the nucleic acid construct into plant cells or plant tissue, and
(iii) a step of culturing the plant cells or plant tissue having the transferred nucleic acid construct to create a plant expressing the nucleic acid construct. This method will be referred to as the “second embodiment”, as appropriate.

### [Third Nucleic Acid Construct]

The nucleic acid construct of step (i) preferably contains all or a portion of the nucleic acid sequence of the SA degradation-associated gene (“third nucleic acid construct” or “SA degradation-associated gene-expressing nucleic acid construct”).

The method for expressing a SA degradation-associated gene in a plant is not particularly restricted, and any method known to a person skilled in the art may be used as appropriate. For example, the method may be one in which an expression cassette is constructed to have a nucleic acid molecule comprising all or a portion of the SA degradation-associated gene linked downstream from the promoter, and the expression cassette is introduced into plant cells. The method of constructing the expression cassette may be selected as appropriate depending on the purpose. For example, a third nucleic acid construct can be constructed by operationally linking a promoter sequence that can be transcribed in plants, a nucleic acid molecule comprising all or a portion of the SA degradation-associated gene, and optionally an appropriate terminator sequence, in that order.

The third nucleic acid construct may be expressed using either a transient expression system or a stable expression system, and may have the following sequences (a) to (e) in order. Sequence (d), however, is optional.
(a) Right border sequence (RB) derived from the Agrobacterium T-DNA sequence;
(b) expression promoter sequence that functions in the plant;
(c) coding sequence of a gene associated with SA degradation;
(d) terminator sequence that functions in the plant;
(e) left border sequence (LB) derived from the Agrobacterium T-DNA sequence.

Step (ii) and step (iii) of the second embodiment are the same as explained for Embodiment 1-1.

### Third Embodiment

The third embodiment of the invention relates to a method for producing a target protein using a plant created by the first embodiment. The third embodiment includes creating a transgenic plant having suppressed or disrupted expression of a gene associated with a resistance mechanism (i.e. a target gene) by the method of Embodiment 1-1 or Embodiment 1-2, and introducing a nucleic acid construct for expression of the target protein in a plant (“fourth nucleic acid construct” or “target protein-expressing nucleic acid construct”) into plant cells or plant tissue, to create a genome-edited plant a transgenic plant, etc., expressing the fourth nucleic acid construct, and a genome-edited plant. In other words, the method uses a transgenic plant, etc., or genome-edited plant having suppressed or disrupted target gene expression to express a target protein in the transgenic plant, etc., or genome-edited plant.

### [Fourth Nucleic Acid Construct]

For example, a fourth nucleic acid construct can be constructed by operationally linking a promoter sequence that can be transcribed in plants, a gene coding for a target protein (exogenous gene), and optionally an appropriate terminator sequence, in that order. Introduction of the fourth nucleic acid construct into a plant may be before step (ii), simultaneously with step (ii) or after step (ii) of the process of Embodiment 1-1 or Embodiment 1-2. That is, the fourth nucleic acid construct may be introduced into the plant separately from the first nucleic acid construct or second nucleic acid construct.

The method of expressing the first nucleic acid construct and fourth nucleic acid construct may be carried out in either a transient expression system or a stable expression system. A transient expression system is preferably used since it allows expression of the target protein in a short period of about 3 days to about 2 weeks. However, since the method of expressing the second nucleic acid construct is a stable expression system, the method of expressing the fourth nucleic acid construct combined with it is likewise in a stable expression system.

The transient expression system for expression of the fourth nucleic acid construct may be agroinfiltration or a plant viral vector, or agroinfection that combines both.

When the fourth nucleic acid construct is expressed in a plant by an agroinfiltration method, the fourth nucleic acid construct may have a right border sequence (RB) and left border sequence (LB) derived from the T-DNA sequence of Agrobacterium at both ends of the T-DNA sequence, as explained above for the method of Embodiment 1-1.

The plant viral vector used for expression of the fourth nucleic acid construct by a plant viral vector is selected from the group consisting of full viral vectors, and deconstructed viral vectors such as tobacco mosaic virus (TMV), plum pox virus (PPV), turnip vein-clearing virus (TVCV), potato virus X (PVX), bean yellow dwarf virus (BeYDV), alfalfa mosaic virus (AIMV), cucumber mosaic virus (CMV), cowpea mosaic virus (CPMV) and zucchini yellow mosaic virus (ZYMV).

An agroinfection method may be a combination of a virus vector and Agrobacterium T-DNA, such as a combination of CMV vector and Agrobacterium T-DNA sequence (see U.S. Patent Application Publication No. 2016/0002654, for example).

When the fourth nucleic acid construct is to be introduced simultaneously with step (ii), the exogenous gene is incorporated into and operationally linked with the first nucleic acid construct, and this is prepared as the fourth nucleic acid construct for transfer. The method of expressing the fourth nucleic acid construct in this case may be carried out in a transient expression system or a stable expression system, but it is preferably carried out in a transient expression system. The exogenous gene may also be incorporated into and operationally linked with the second nucleic acid construct to create the fourth nucleic acid construct, but in this case the method of expressing the fourth nucleic acid construct is carried out in a stable expression system.

The exogenous gene used in the method for producing a target protein is not particularly restricted, and any gene may be used. Examples include natural and synthetic genes, as well as their fragments, mutants and modified forms.

Specific examples of exogenous genes include cytokines, immunogenic substances, antibodies, enzymes, blood-derived components, adjuvant agents, virus-derived components and pathogenic microorganism-derived components.

Step (ii) and step (iii) of the third embodiment are the same as explained for Embodiment 1-1.

### Fourth Embodiment

The fourth embodiment of the invention relates to a method for producing a target protein using a plant created by the second embodiment. The fourth embodiment includes creating a transgenic plant, etc., having overexpression of a SA degradation-associated gene by the method of the second embodiment, and introducing the fourth nucleic acid construct into plant cells or plant tissue to create a transgenic plant, etc., expressing the fourth nucleic acid construct. That is, the method causes high expression of a target protein in a plant by using a transgenic plant, etc., having overexpression of a SA metabolism-associated gene.

Introduction of the fourth nucleic acid construct into a plant in the fourth embodiment may be before step (ii), simultaneously with step (ii) or after step (ii) of the process of the second embodiment. That is, the fourth nucleic acid construct may be introduced into the plant separately from the third nucleic acid construct. When the fourth nucleic acid construct is to be introduced simultaneously with step (ii), the exogenous gene is incorporated into and operationally linked with the third nucleic acid construct, and this is prepared as the fourth nucleic acid construct for transfer. The expression method may be in a transient expression system or a stable expression system.

The structure of the fourth nucleic acid construct of the fourth embodiment and the creation method are the same as explained for the third embodiment. The transient expression system is also the same as explained for the third embodiment.

### Fifth Embodiment

The fifth embodiment of the invention relates to a method for producing a target protein using a plant. The fifth embodiment includes creating a transgenic plant, etc., obtained by the method of Embodiment 1-1 or the second embodiment, or a genome-edited plant obtained by the method of Embodiment 1-2, and introducing the fourth nucleic acid construct into plant cells or plant tissue to create a transgenic plant, etc., or genome-edited plant, expressing the fourth nucleic acid construct. With this method it is possible to introduce the fourth nucleic acid construct into Agrobacterium, for example, and to further introduce this into a transgenic plant, etc., obtained by the method of Embodiment 1-1 or the second embodiment. It is also possible to introduce the fourth nucleic acid construct into Agrobacterium, for example, and to further introduce this into a genome-edited plant obtained by the method of Embodiment 1-2.

The structure of the fourth nucleic acid construct of the fifth embodiment and the creation method are the same as explained for the third embodiment.

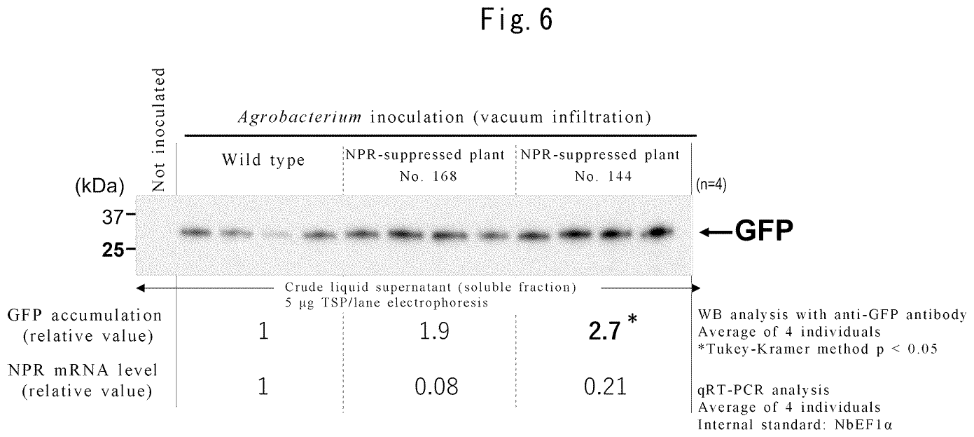

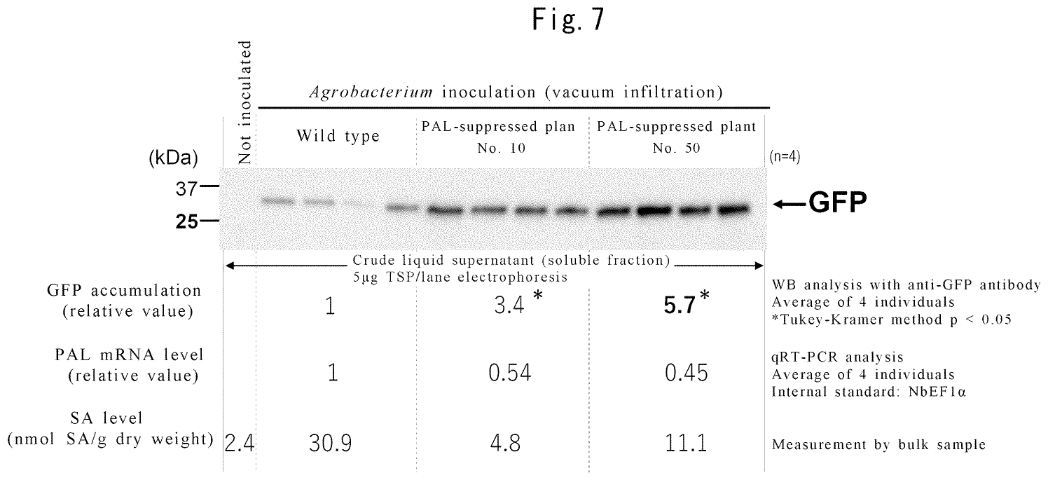

The method of expressing the fourth nucleic acid construct for the fifth embodiment may be carried out in either a transient expression system or a stable expression system. For example, FIGS. 6 and 7 show examples of expression of a target protein (GFP) in a transient expression system using an NPR gene suppressed plant and a PAL gene suppressed plant, each created in a stable expression system.

By using the method of expressing the fourth nucleic acid construct for the fifth embodiment, it is expected to be possible to satisfactorily express not only GFP but also various cytokines, immunogenic substances, antibodies, enzymes, etc.

The transient expression system is also the same as explained for the third embodiment.

### Sixth Embodiment

The sixth embodiment of the invention relates to a transgenic plant, etc., obtained by the method of Embodiment 1-1, and a genome-edited plant obtained by the process of Embodiment 1-2. In particular, in a transgenic plant with suppressed expression of the NPR gene and a transgenic plant with suppressed expression of the PAL gene, the accumulated amounts of the target gene mRNA are reduced compared to the wild type plant. In a transgenic plant with suppressed expression of the PAL gene, it was confirmed that the amount of biosynthesized SA (salicylic acid) was reduced. On the other hand, in a transgenic plant with suppressed expression of the NPR gene, it was confirmed that expression of PR (pathogenesis related) genes downstream from the NPR gene was suppressed compared to the wild type plant.

### Seventh Embodiment

The seventh embodiment of the invention relates to a transgenic plant, etc., obtained by the method of the second embodiment. In particular, in an SGT-transformed plant, SGT was overexpressed compared to the wild type plant, with GFP being detected only in the leaf veins of the wild type plant. In the SGT-transformed plant, GFP was also detected in mesophyll cells in addition to the leaf veins, and more accumulation of the target protein (GFP) was confirmed compared to the wild type plant. This suggests that resistance was suppressed by SGT overexpression.

### Eighth Embodiment

The eighth embodiment of the invention relates to a transgenic plant, etc., or genome-edited plant obtained by the method of the third to fifth embodiments. In particular, in a transgenic plant with suppressed expression of the NPR gene and a transgenic plant with suppressed expression of the PAL gene, the target protein (GFP) was confirmed to be more highly expressed compared to the wild type plant.

In a transgenic plant with suppressed expression of the EDS1 gene and a transgenic plant with suppressed expression of the PAD4 gene as well, which were created by the VIGS method, it was confirmed that the level of biosynthesized SA was reduced and the target protein (GFP) was more highly expressed, compared to the wild type plant.

Since all of the other genes associated with resistance mechanisms are genes associated with resistance mechanisms in plants, it is possible to create transgenic plants or genome-edited plants with suppression or disruption of the genes, or transgenic plants with overexpression of the genes, thus making it possible to highly express the target proteins.

Moreover, by cross-breeding any two transgenic plants or genome-edited plants from transgenic plants with suppressed expression of a gene associated with the SA biosynthetic pathway, obtained by the method of Embodiment 1-1, genome-edited plants with disrupted expression of a gene associated with the SA biosynthetic pathway, obtained by the method of Embodiment 1-2, transgenic plants with high expression of a gene associated with SA degradation, obtained by the method of the second embodiment, transgenic plants with suppressed expression of a gene associated with SA signal transduction, obtained by the method of Embodiment 1-1, and genome-edited plants with disrupted expression of a gene associated with SA signal transduction, obtained by the method of Embodiment 1-2, it is possible to create plants having both traits.

According to the invention, it is possible to create a plant with suppressed pathogen resistance, and to achieve high expression of a target protein by using the plant. This indicates that it is possible to significantly increase productivity of desired useful substances by suppressing the resistance mechanism inherent in plants.

### EXAMPLES

The present invention will now be explained in greater detail by the following Examples. It is to be understood, however, that the invention is not restricted in any way to the Examples, and various modifications thereof may be implemented.

### [Example 1] Construction of Plant Expression Vector with Inverted Repeat Structure of NPR Gene

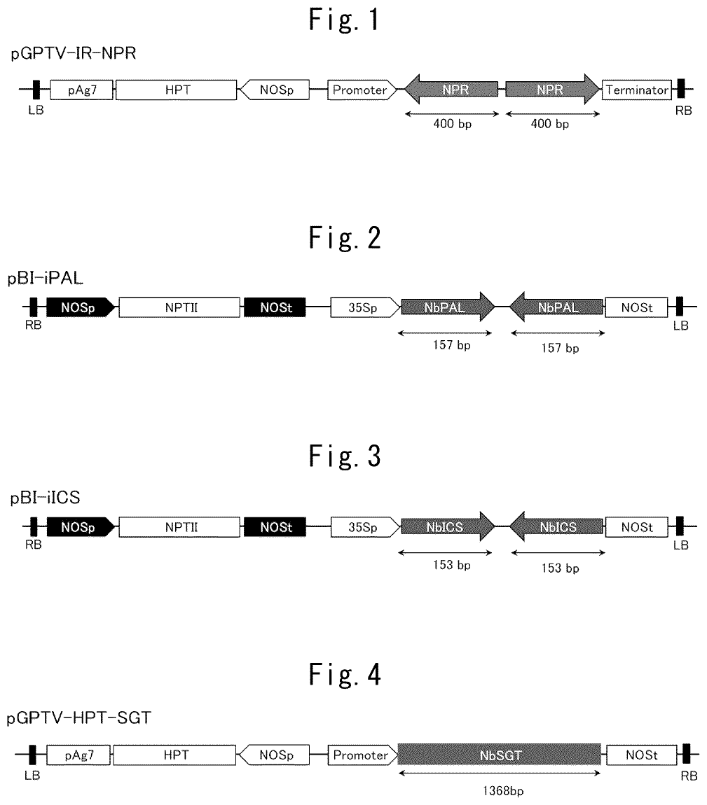

An inverted repeat sequence (IR-NPR) having a partial sequence of the NPR gene encoded by Nicotiana benthamiana was inserted between the SVBV virus-derived promoter and the terminator, which had been inserted into a binary vector for plant expression having a hygromycin resistance gene expression cassette (pGPTV-HPT) (Becker et al., Plant Molecular Biology, 20: 1195-1197, 1992). In the IR-NPR, a partial sequence of 400 bp (SEQ ID NO: 1) that is able to simultaneously target the NPRa (Niben101Scf14780g01001) gene and the NPRb (Niben101Scf11512g01004) gene was synthesized with the antisense strand and sense strand arranged in that order. The gene numbers are assigned in the following database (N. benthamiana Genome v1.0.1.): https://solgenomics.net/organism/Nicotiana_benthamiana/genome. The structure of the IR-NPR plant expression vector (pGPTV-IR-NPR) is shown schematically in FIG. 1.

SEQ ID NO: 1: NPR gene partial sequence of 400 bp (the following sequence is a sense strand sequence)

GAATGACATCAGCGGAAGCAGTAGTATATGCTGCATCGGCGGCGGCATG
ACAGAATCATTCTCGCCGGAAACTTCGCCGGCAGAGATTACTTCACTGA
AACGCCTCTCTGAAACATTGGAATCTATCTTCGATGCGGCTTCTCCGGA
GTTTGACTACTTCGCCGACGCTAAGCTTGTGATTCCCGGCGCCGGTAAG
GAAATTCCGGTTCACCGGTGCATTTTGTCGGCGAGGAGTCCGTTCTTTA
AGAATTTGTTCTGCGGGAAAAAGGAGAAGAATAGTAATAAGGTGGAATT
AAAGGAAATAATGAAAGAGTATGAAGTGAGCTATGATGGTGTGGTGAGT
GTGTTGGCCTATTTGTATAGTGGAAAAATTAGGCCTTCACCTAAAGATG
TGTGTGTT

### [Example 2] Method of Transferring Plant Expression Vector into Agrobacterium

The freeze-thaw method with liquid nitrogen (Holsters M. et. al., (1978), Mol. Gen. Genet., 163(2):181-7) was used for transformation of the plant expression vector into Agrobacterium tumefaciens LBA4404 (Clontech). The obtained transformed Agrobacterium cells (LBA4404) were permeation-cultured overnight at 28° C. in LB medium with addition of 100 mg/l rifampicin, 300 mg/l streptomycin and 50 mg/l kanamycin, to obtain a cell solution.

### [Example 3] Creation of NPR-Suppressed Plant

Transformation in Nicotiana benthamiana was carried out by the leaf disc method LBA4404 (Horsch R B. et al. (1984), Science, 223:496-498) using Agrobacterium tumefaciens. Leaf discs with diameters of about 1 cm were cut out from tobacco leaves and immersed in LBA4404 cell solution having pGPTV-IR-NPR, and co-culturing was carried out on MS (Murashige-Skoog) agar medium for 2 days. After 3 days, the adhered Agrobacterium was washed off from the co-cultured leaf discs, and the washed co-cultured leaf discs were subcultured in MS solid medium (containing 3% sucrose) with addition of 1 mg/l BAP (6-benzylaminopurine), 0.1 mg/l NAA (naphthaleneacetic acid), 15 mg/l hygromycin and 50 mg/l carbenicillin. After inducing shoots, roots were induced with MS solid medium (containing 3% sucrose) with addition of 15 mg/l hygromycin and 500 mg/l carbenicillin, to obtain multiple lines of cultured plants. The lines of these plants were selected by the method in Example 4 described below, and then the cultured plants of the selected lines were conditioned in a closed recombinant greenhouse and grown by soil cultivation to obtain next-generation seeds (T1 individuals).

### [Example 4] Analysis of NPR Expression Suppression in Candidate Plant

After liquid nitrogen freezing of fresh leaves of the rooted cultured plant created in Example 3 or fresh leaves of the agroinfiltration inoculated plants of Example 15, they were ground, and the total RNA was extracted by the AGPC extraction method (Chomczynski, P. et al., (1987), Anal. Biochem. 162:156-159). The total RNA was subjected to DNase treatment and cDNA was synthesized by reverse transcription using random primers, and then real-time PCR was carried out using a LightCycler 96 system (Roche Diagnostics). For analysis of NPR gene expression level, there were used primers able to simultaneously detect the NPRa (Niben101Scf14780g01001) gene and NPRb (Niben101Scf11512g01004) gene (SEQ ID NOs: 2 and 3), and a hydrolysis probe (Universal ProbeLibrary Probe #108, Roche Diagnostics). As an internal standard, Nicotiana benthamiana elongation factor 1α (Nb EF1α; GenBank accession number AY206004) was used for quantification. EF1α (elongation factor 1α) gene was detected using primers (SEQ ID NOs: 4 and 5) and a hydrolysis probe (Universal ProbeLibrary Probe #56, Roche Diagnostics).

### Primers for Detection of NPR Genes (NPRa, NPRb)

SEQ ID NO: 2:
TGTGTGTGTTTGTGTGGACAAT (22 mer) (forward)
SEQ ID NO: 3:
GAACGCTACAGCTGGCCTAC (20 mer) (reverse)
EF1α gene detection primers SEQ ID NO: 4:
CTGGTACCTCCCAAGCTGAC (20 mer) (forward)
SEQ ID NO: 5:
CCAGCTTCAAAACCACCAGT (20 mer) (reverse)

### [Example 5] Construction of Plant Expression Vector with Inverted Repeat Structure of PAL Gene

An inverted repeat sequence (iPAL) having a partial sequence of the PAL gene encoded by Nicotiana benthamiana was inserted between the virus-derived promoter and the terminator, which had been already inserted into a binary vector for plant expression having a kanamycin resistance gene expression cassette (pBI121) (Jefferson et al., The EMBO Journal, vol. 6, no. 13, pp. 3901-3907 (1987)). In iPAL, the PAL gene partial sequence of 157 bp (SEQ ID NO: 6) that can simultaneously target multiple PAL genes (Niben101Scf12881g00009, Niben101Scf12881g00010, Niben101Scf05442g03015, Niben101Scf04652g00007, Niben101Scf04090g02003, Niben101Scf05617g00005, Niben101Scf04375g02015, Niben101Scf03712g01008, Niben101Scf02432g00011) was synthesized with the sense strand and antisense strand arranged in that order. FIG. 2 schematically shows the structure of an iPAL plant expression vector (pBI-iPAL). The vector was introduced into Agrobacterium by the method described in Example 2.

SEQ ID NO: 6: PAL gene partial sequence of 157 bp (the following sequence is a sense strand sequence) accaaagcaagatcgttacgccctcagaacatcaccccagtggcttggccctcaaattgaggtcatccgttctgcaaccaagatgattgaga gagagattaactcagtgaacgacaaccctttgatcgatgtttcaagaaacaaggcgttacacggt

### [Example 6] Creation of PAL-Suppressed Plant

Transformation in Nicotiana benthamiana was carried out by the leaf disc method LBA4404 (Horsch R B. et al., (1984), Science, 223:496-498) using Agrobacterium tumefaciens. Leaf discs with diameters of about 1 cm were cut out from tobacco leaves and immersed in LBA4404 cell solution having pBI-iPAL, and co-culturing was carried out on MS (Murashige-Skoog) agar medium for 2 days. After 3 days, the adhered Agrobacterium was washed off from the co-cultured leaf discs, and the washed co-cultured leaf discs were subcultured in MS solid medium (containing 3% sucrose) with addition of 1 mg/l BAP (6-benzylaminopurine), 0.1 mg/l NAA (naphthaleneacetic acid), 50 mg/l kanamycin and 500 mg/l carbenicillin. After inducing shoots, roots were induced with MS solid medium (containing 3% sucrose) with addition of 50 mg/l kanamycin and 500 mg/l carbenicillin, to obtain multiple lines of cultured plant. The lines of these plants were selected by the method described in Example 7 described below, and then the cultured plants of the selected lines were conditioned in a “closed” recombinant greenhouse and grown by soil cultivation to obtain next-generation seeds (T1 individuals).

### [Example 7] Analysis of PAL Expression Suppression in Candidate Plants

After liquid nitrogen freezing of fresh leaves of the rooted cultured plant created in Example 6 or fresh leaves of the agroinfiltration inoculated plant of Example 15, they were ground, and the total RNA was extracted by the AGPC extraction method (Chomczynski, P. et al., (1987), Anal. Biochem. 162:156-159). The total RNA was subjected to DNase treatment and cDNA was synthesized by reverse transcription using random primers, and then real-time PCR was carried out using a LightCycler 96 system (Roche Diagnostics). For analysis of PAL gene expression level there were used primers (SEQ ID NOs: 7 and 8) and a hydrolysis probe (Universal ProbeLibrary Probe #142, Roche Diagnostics). Nb EF1α was used as an internal standard for quantification. Detection of the EF1α gene was carried out using the same primers as the primers described in Example 4 (SEQ ID NOs: 4 and 5) and the same hydrolysis probe.

### PAL Gene Detection Primers

SEQ ID NO: 7:
TGCCATGGCTTCATACTGTT (20 mer) (forward)
SEQ ID NO: 8:
CGGCACTTTGTACGTGGTTA (20 mer) (reverse)

### [Example 8] Construction of Plant Expression Vector with Inverted Repeat Structure of ICS Gene

An inverted repeat sequence (iICS) having a partial sequence of the ICS gene encoded by Nicotiana benthamiana was inserted between the virus-derived promoter and the terminator, which had been inserted into a binary vector for plant expression having a kanamycin resistance gene expression cassette (pBI121). In the iICS, a partial sequence of 153 bp (SEQ ID NO: 9) of the ICS gene that is able to simultaneously target the ICSa gene (Niben101Scf00593g04010) and the ICSb gene (Niben101Scf05166g06006) was synthesized with the sense strand and antisense strand arranged in that order. FIG. 3 schematically shows the structure of an iICS plant expression vector (pBI-iICS). The vector was introduced into Agrobacterium by the method described in Example 2.

SEQ ID NO: 9: ICS gene partial sequence of 153 bp (the following
sequence is a sense strand sequence).
CCAGAGGTCAATAGAAGCACTTCAGGCCACAATATGGCAGGTTTCCTCCGTTCTTAT
GAGGGTGCAGAAAAAAATATCTCGTTCACATATACTCGCGAGTACTCATGTCCCGG
GTAAAGCATCTTGGGACCAAGCTGTTAAGCGTGCTTTGCA

### [Example 9] Creation of ICS-Suppressed Plants

Transformation in Nicotiana benthamiana was carried out by the leaf disc method LBA4404 (Horsch R B. et al., (1984), Science, 223:496-498) using Agrobacterium tumefaciens. Leaf discs with diameters of about 1 cm were cut out from leaves and immersed in LBA4404 cell solution having pBI-iICS, and co-culturing was carried out on MS (Murashige-Skoog) agar medium for 2 days. After 3 days, the adhered Agrobacterium was washed off from the co-cultured leaf discs, and the washed co-cultured leaf discs were subcultured in MS solid medium (containing 3% sucrose) with addition of 1 mg/l BAP (6-benzylaminopurine), 0.1 mg/l NAA (naphthaleneacetic acid), 50 mg/l kanamycin and 500 mg/l carbenicillin. After inducing shoots, roots were induced with MS solid medium (containing 3% sucrose) with addition of 50 mg/l kanamycin and 500 mg/l carbenicillin, to obtain multiple lines of cultured plants. The lines of these plants were selected by the method described in Example 10, and then the cultured plants of the selected lines were conditioned in a “closed” greenhouse and grown by soil cultivation to obtain next-generation seeds (T1 individuals).

### [Example 10] Analysis of ICS Expression Suppression in Candidate Plants

After liquid nitrogen freezing of fresh leaves of the rooted cultured plant created in Example 9 or fresh leaves of the agroinfiltrated (inoculated) plants of Example 16, they were ground, and the total RNA was extracted by the AGPC extraction method (Chomczynski, P. et al., (1987), Anal. Biochem. 162:156-159). The total RNA was subjected to DNase treatment and cDNA was synthesized by reverse transcription using random primers, and then real-time PCR was carried out using a LightCycler 96 system (Roche Diagnostics). For analysis of ICS gene expression level there were used primers (SEQ ID NOs: 10 and 11) and a hydrolysis probe (Universal ProbeLibrary Probe #63 Roche Diagnostics). Nb EF1α was used as an internal standard for quantification. Detection of the EF1α gene was carried out using the primers described in Example 4 (SEQ ID NOs: 4 and 5) and the same hydrolysis probe.

### ICS Gene Detection Primers

SEQ ID NO: 10:
ATTCCGCCATCTCTGACTTG (20 mer) (forward)
SEQ ID NO: 11:
GGCACCTCAAGACGAATGATA (21 mer) (reverse)

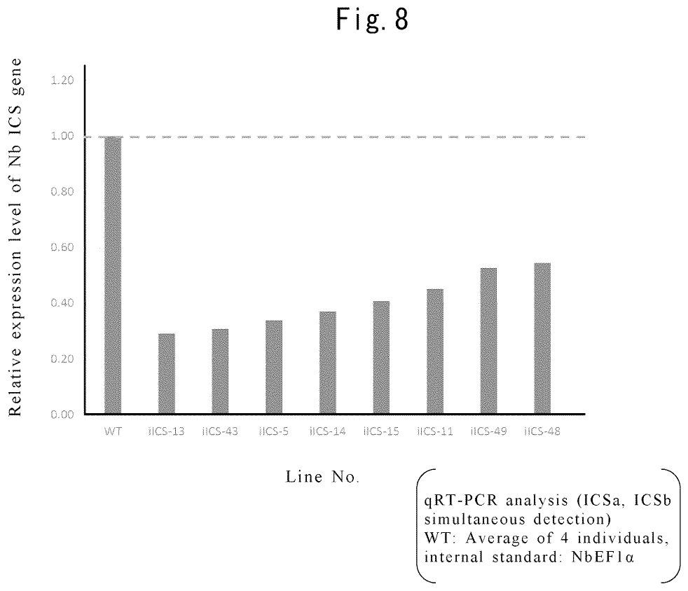

Upon comparing the expression level of the Nicotiana benthamiana ICS gene in the wild type (WT) plant with the expression levels of the created ICS-suppressed plants, four lines (Nos. 13, 43, 5, 14) were obtained as individuals with expression levels reduced to 40% or lower. FIG. 8 shows the results of RT-PCR analysis of ICS gene expression levels.

### [Example 11] Construction of Plant Expression Vector Overexpressing SGT Gene

The SGT gene of 1371 bp (SEQ ID NO: 12) of Nicotiana benthamiana (Nb) was inserted downstream from the internal sequence of strawberry vein binding virus (SVBV)-derived promoter which had already been inserted into a binary vector for plant expression having the hygromycin resistance gene expression cassette (pGPTV-HPT). Specifically, PCR was conducted using Nicotiana benthamiana cDNA as template, and using a primer providing the restriction enzyme site (SEQ ID NO: 13) and a primer providing the stop codon and restriction enzyme site (SEQ ID NO: 14) at both ends of the SGT gene (1368 bp having 99.85% homology with Niben101Scf00788g02014), to obtain an amplification product. An XbaI-SGT gene-SacI fragment having a restriction enzyme sequence added by TA cloning was ligated onto the vector to construct pGPTV-HPT-SGT. The cDNA clone sequence was analyzed to confirm the restriction enzyme site, and sequenced using ABI PRISM Big Dye Terminator (Applied Biosystems, USA), to confirm the absence of mismatches. The structure of the constructed plant expression vector (pGPTV-HPT-SGT) is shown schematically in FIG. 4. The vector was introduced into Agrobacterium by the method described in Example 2.

Similarly, the NbSGT gene was also inserted downstream from the 35S promoter of a binary vector (pBI121) for plant expression having the kanamycin resistance gene expression cassette, to create pBI121-SGT.

SEQ ID NO: 12: SGT gene partial sequence of 1371 bp (the following
sequence isa sense strand sequence).
ATGACTACTCACAAAGCTCATTGCTTGATCTTGCCATATCCAGCCCAAGGTCATATC
AACCCTATGCTCCAATTCTCCAAACGTTTGCAATCCAAAGGTGTGAAAATCACTATT
GCAGCGACCAAATCCTTCTTGAAAAACATGCAAGAGTTGTCAACTTCTGTGTCAGTC
GAGGCTATCTCCGATGGCTATGATGATGGTGGCCGTGAGCAAGCTGGAACCTTTGTG
GCCTATTTTACAAGATTCAAAGAAGTTGGCTCGGATACTCTGTCTCAGCTTATTGAA
ACGTTAATGATTCGTGGTTGTCCTGTAAATTGCATTGTTTACGATCCATTTCTTCCTT
GGGCTGTAGAAGTAGCAAAGAAGTTTGGATTAGCTACTGCTGCTTTTTTCACACAAT
CTTGTACAGTGGATAACATTTATTACCATGTACATAAAGGGGTTCTAAAACTTCCTC
CAACTGATGTTGATGAAGAAATCTCAATTCCTGGATTATTAACAGTTGAGACATCAG
ATGTACCTACTTTTGAAATTAATCCTGAATCAACAAGAGTTCTTGAAATGTTGGTGA
ATCAGTTCTCGAATCTTGATAAAGTGGATTGGGTTCTAATCAACAGCTTCTATGAAT
TGGAGAAAGAGGTAATTGATTGGATGTCCGAGGTTTATCCAGTCAAGACAATTGGA
CCAGTTATACCATCAATGTACTTAGACAAGAGGCTACTAGATGATAAAGAATATGG
CCTTAGTGTCTTCAAACCAATGACAGATGCATGTCTAAACTGGTTAAACCATCAACC
AGCTAGCTCAGTAGTATATGTATCATTTGGAAGCTTAGCCAAATTAGAAGCAGAAC
AAATGGAAGAAGTAGCATGGGGTTTGAGAAATAGCAACAAGAACTTCTTATGGGTA
GTTAGATCCACTGAAAAATCCAAACTTCCCAAGAACTTTGTAGAGGAATTAGCAAG
TGAAAAAGGCTTAGTGGTGTCATGGTGTCCACAATTACAAGTCTTGGAACACAAATC
AATCGGGTGTTTTCTCACGCACTGTGGGTGGAATTCGACTTTAGAAGCGATTAGTTT
GGGAGTAACAATGGTTGCGATGCCGCAATGGTCAGGTCAACCTACAAATGCAAAGC
TTGTGAAGGATATTTGTGAGATGGGTGTTAGAGCAAAACAAGATGAGAAAGGGATT
GTTAGAAGAGAAATTATTGAAGAATATATAAAGATAGTAATGGAAGAAGAGAAAG
GAAAAATGATTAGGAAAAGTGCAAAGAAATGGAAGGAATTGGCTAGAAAAGCTGT
GGATGAAGGAGGAAGTTCAGATAGAAATATTGAAGAATTTGTTTCCAAGTTGATGA
CTATTTCTTAATAG

### SGT Gene Amplification Primers

SEQ ID NO: 13:
CCTCTAGAATGACTACTCACAAAGCTCATTG (31 mer) (forward)
SEQ ID NO: 14:
TGAGCTCCTATTAAGAAATAGTCATCAACTTG (32 mer) (reverse)

### [Example 12] Creation of SGT-Overexpressing Plant

Transformation in Nicotiana benthamiana was carried out by the leaf disc method LBA4404 (Horsch R B. et al., (1984), Science, 223:496-498) using Agrobacterium tumefaciens. Leaf discs with diameters of about 1 cm were cut out from tobacco leaves and immersed in LBA4404 cell solution which had the plant expression vector, and co-culturing was carried out on MS (Murashige-Skoog) agar medium for 2 days. After 3 days, the adhered Agrobacterium was washed off from the leaf discs co-cultured with the cells which had pGPTV-HPT-SGT, and the washed leaf discs were subcultured in MS solid medium (containing 3% sucrose) with addition of 1 mg/l BAP (6-benzylaminopurine), 0.1 mg/l NAA (naphthaleneacetic acid), 15 mg/l hygromycin and 50 mg/l carbenicillin. After inducing shoots, roots were induced with MS solid medium (containing 3% sucrose) with addition of 15 mg/l hygromycin and 50 mg/l carbenicillin, and then the cultured individuals were conditioned in a “closed” greenhouse and grown by soil cultivation, to obtain next-generation seeds (T1 individuals).

After 3 days, the adhered Agrobacterium was washed off from the leaf discs co-cultured with cells that had pBI121-SGT, and the washed leaf discs were subcultured in MS solid medium (containing 3% sucrose) with addition of 1 mg/l BAP (6-benzylaminopurine), 0.1 mg/l NAA (naphthaleneacetic acid), 50 mg/l kanamycin and 500 mg/l carbenicillin. After inducing shoots, roots were induced with MS solid medium (containing 3% sucrose) with addition of 50 mg/l kanamycin and 500 mg/l carbenicillin, to obtain multiple lines of cultured plants. The lines of these plants were selected by the method described in Example 13, and then the cultured plants of the selected lines were conditioned in a “closed” greenhouse and grown by soil cultivation to obtain next-generation seeds (T1 individuals).

### [Example 13] Analysis of Expression of SGT Protein by SGT Transformants

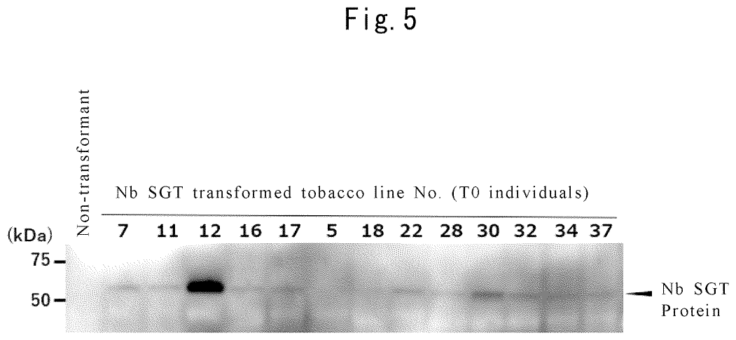

Fresh leaves were harvested from the cultured plant created by the method described in Example 12 (T0 individuals) and crushed using liquid nitrogen, after which PBS was added and the crushed leaves were ground. The ground solution was centrifuged (4° C., 15,000 rpm, 10 min), and then the supernatant was mixed with SDS-PAGE electrophoresis buffer and the mixture was provided for SDS-PAGE. For transfer, a PVDF membrane was used for Western blot analysis using anti-SGT antibody. Transformed tobacco individuals expressing SGT protein were selected from the regenerated cultured individuals. FIG. 5 shows a photograph of the Western blot analysis results. In this Example, multiple lines were obtained in which high expression of SGT was confirmed compared to the non-transformants (wild type plant).

The recombinant plants created in Examples 1 to 13 were analyzed by the following methods in Examples 14 to 21.

### [Example 14] Confirming Exogenous Gene Transfer in Candidate Plant (Plant Expression Vector Transfer)

After sampling leaves of the rooted cultured plant created in Example 3, Example 6, Example 9 and Example 12, an Extract-N-Amp Plant PCR Kit (Sigma, cat.XNA-P2) was used to prepare a leaf lysate, and Extract N-Amp PCR kit reaction mix was used for PCR to amplify the part of the plant expression vector construct transferred into the cultured plant. The primer sequences used are shown below. Since it is expected that individuals for which PCR amplification was confirmed had the introduced plant expression vector construct successfully inserted into the chromosomes, the cultured individuals were established as transformed benthamiana lines.

### Detection of Transgenes in NPR-Suppressed Plant

SEQ ID NO: 15:
TGGCCAGCTAGCTATCACTGAAAAG (25 mer) (forward)
SEQ ID NO: 16:
CCTGGGGTCAGTCTTATCTTCAGC (24 mer) (reverse)

### Detection of Transgenes in PAL-Suppressed Plant

SEQ ID NO: 17:
CCTTCGCAAGACCCTTCCTC (20 mer) (forward)
SEQ ID NO: 18:
TTGAAACATCGATCAAAGGG (20 mer) (reverse)

### Detection of Transgenes in ICS-Suppressed Plant SEQ ID NO: 17: Same as Above

SEQ ID NO: 19:
TTGGTCCCAAGATGCTTTAC (20 mer) (reverse)

### Detection of Transgenes in SGT-Overexpressing Plant

For Transgene pGPTV-HPT-SGT:

SEQ ID NO: 20:
CATTTGAAGGCAGAGGCGAACAC (23 mer) (forward)
SEQ ID NO: 21:
ACTGAGCTAGCTGGTTGATGG (21 mer) (reverse)

For Transgene pBI121-SGT:
SEQ ID NO: 17: Same as above
SEQ ID NO: 21: Same as above

### [Example 15] Agroinfiltration (Vacuum Infiltration) Method

The agroinfiltration method used was a modification of the method described in the literature (Grimsley N. et. al., (1986), Proc. Natl. Acad. Sci. USA. 83:3282-3286). Agrobacterium harboring the GFP gene was transformed by the method described in Example 2 to obtain a recombinant Agrobacterium cell solution. The recombinant Agrobacterium cell solution was centrifuged (20° C., 6,000 rpm, 15 min) and the obtained cell pellet was suspended in MES buffer (final concentration: 10 mM MES, 10 mM MgCl₂, pH 5.7) to OD600=0.6. Since a mixture of several types of bacterial cell solutions is used when inoculating a CMV-agroinfection vector, they were mixed to a final concentration of OD600=0.6 for each bacterial cell solution (see Japanese Patent No. 6350995). The bacterial cell solutions were forcibly injected into a wild type plant, an NPR-suppressed plant, a PAL-suppressed plant and an SGT-high-expressing plant, using a vacuum desiccator.

### [Example 16] Cultivation of Bacteria-Inoculated Plants

The plants of Examples 3, 6 and 12, which had transient expression of the target protein (GFP) by agroinfiltration (vacuum infiltration) by the method described in Example 15, were cultivated at 23° C. with a 16 hour-light period and a 8-hour-dark period, in an artificial weather apparatus or lighted plant cultivating incubator.

### [Example 17] Western Blotting for Detection of Target Protein (GFP) in Inoculated Plants

Leaves were harvested from plant transiently expressing GFP, obtained in Example 15 and Example 16, and crushed using liquid nitrogen, after which Tris buffer was added and the leaves were further ground.

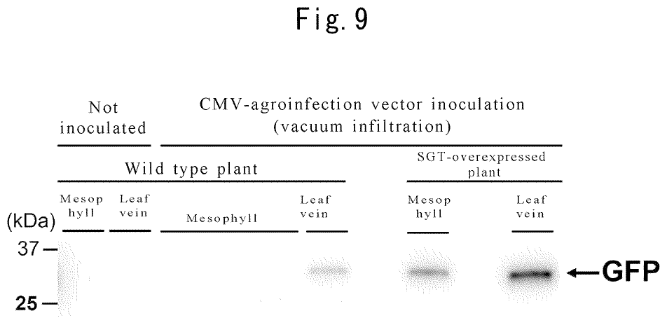

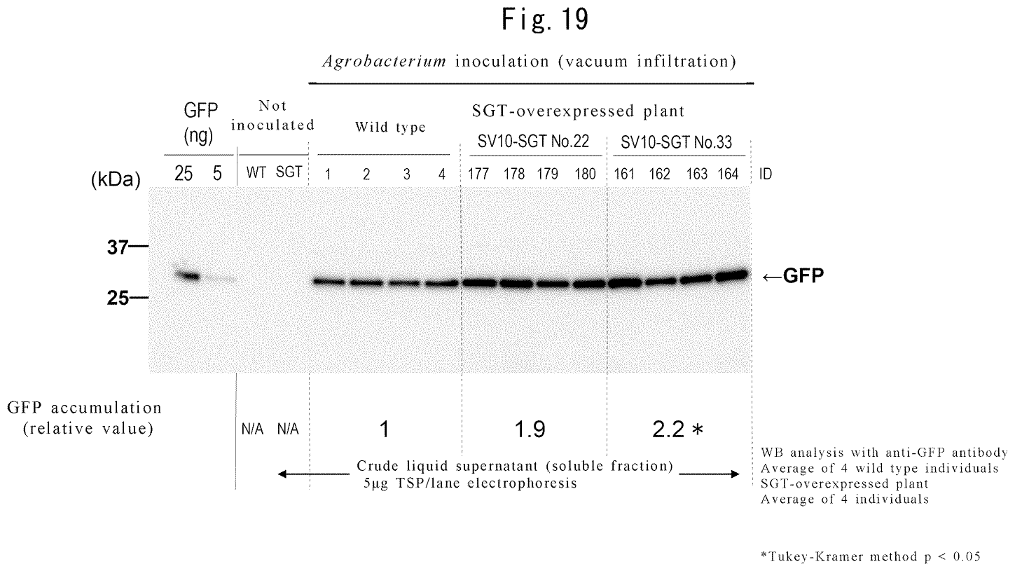

The ground solution was centrifuged (15,000 rpm, 4° C., 10 min), and then the crude supernatant was collected. The total amount of protein (TSP) was quantified by the BCA method, and for Western blot analysis, each lane was adjusted to a fixed amount of TSP, electrophoresed in 12% SDS-PAGE gel, and then transferred to a PVDF membrane. Immunostaining carried out with a specific antibody (anti-GFP antibody) was followed by chemiluminescence detection using ChemiDoc (Bio-Rad), for quantification of the band strength. FIG. 6 shows the results of transient expression of the target protein (GFP) by agroinfiltration in an NPR-suppressed plant, FIG. 7 shows the results of transient expression of the target protein (GFP) by agroinfiltration in a PAL-suppressed plant, FIG. 19 shows the results of transient expression of the target protein (GFP) by agroinfiltration in an SGT-overexpressing plant, and FIG. 9 shows the results of transient expression of the target protein (GFP) by agroinfection in an SGT-overexpressing plant. The GFP expression amounts were confirmed to be increased in the NPR-suppressed plant (FIG. 6), the PAL-suppressed plant (FIG. 7) and the SGT-overexpressing plant (FIG. 19), compared to the wild type plant. In the wild type plant of FIG. 9, GFP was only detected in the leaf veins, whereas in the SGT-overexpressing plant, GFP was detected in both the leaf veins and mesophyll, indicating a wider range of expression. In other words, GFP expression level was confirmed to have increased in the SGT-overexpressing plant.

### [Example 18] GFP Detection in Leaves of Inoculated Plants

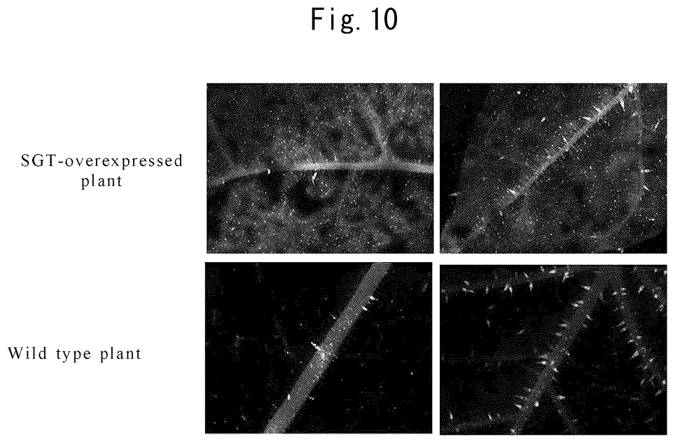

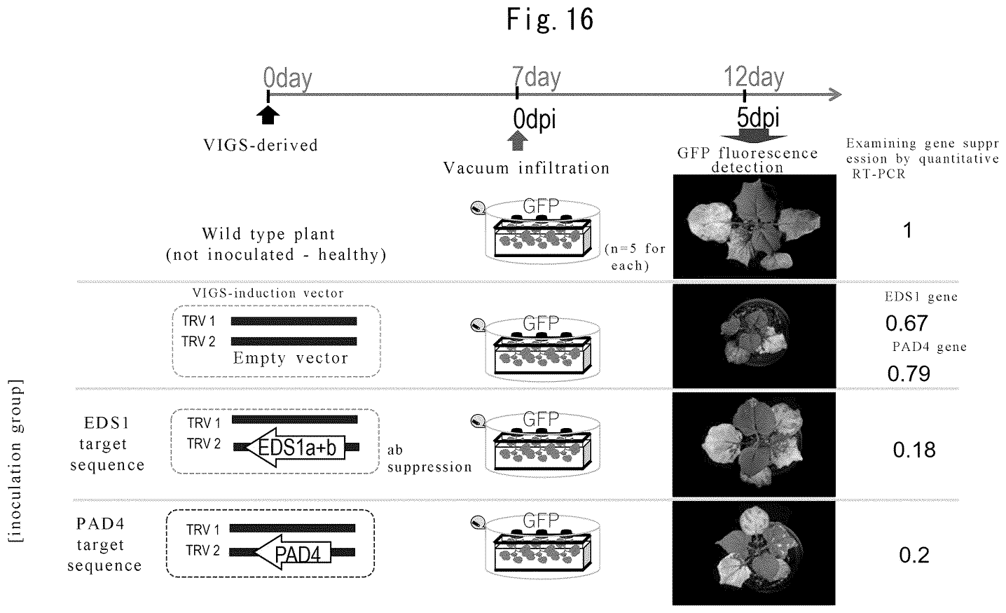

In order to confirm target gene expression in inoculated plants, the GFP inoculated individuals were irradiated with blue light in a visualizable wavelength range, and a filter was used to observe GFP fluorescence in the plants. A GFP fluorescence photograph was obtained using a GFP SZX-RFL2 (SZX fluorescence illumination system: Olympus Corp.) or a digital camera. FIG. 10 is a photograph showing GFP fluorescence in the leaves of the SGT-transformed N. benthamiana (SGT-overexpressing plant obtained in Example 9 (top photographs) and GFP fluorescence in the leaves of wild type N. benthamiana (wild type plant) (bottom photographs). FIG. 16 shows photographs of GFP fluorescence in the leaves of a VIGS-induced EDS1-suppressed plant obtained in Example 28, and a VIGS-induced PAD4-suppressed plant obtained in Example 29.

### [Example 19] Statistical Analysis

Data for each individual were analyzed in a multiple comparison test by the Tukey-Kramer method. Statistical testing was conducted with a significance level of 5%. The results are shown at the bottom in FIGS. 6, 7, 14 and 15. In the diagrams, “*” indicates a group showing a significant difference compared to the wild type plant.

### [Example 20] Extraction of Salicylic Acid and Salicylic Acid Glycoside from Leaves

The salicylic acid and salicylic acid glycoside were extracted by a modification of the method of Tugizimana et al. (Metabolites 9:194 (2019)).

After first adding 80% methanol containing 0.1% formic acid to the powdered freeze-dried leaves and vortexing for 5 minutes, sonication was carried out for 20 minutes at ice temperature to extract the salicylic acids, after which centrifugation was carried out at 12,000 rpm for 5 minutes and the supernatant was transferred to a fresh tube. The supernatant collected after repeating this procedure twice was allowed to stand for 1 hour at ice temperature and centrifuged at 6,000 rpm for 10 minutes, and then an Oasis PRiME HLB column (Waters) was used to purify the supernatant.

### [Example 21] LCMS Analysis of Salicylic Acid and Salicylic Acid Glycoside

The salicylic acid and salicylic acid glycoside extracted in Example 20 were analyzed by LCMS of salicylic acid and salicylic acid glycoside using a modification of the method of Pastor et al. (Plant Physiology and Biochemistry 53: 19 (2012)).

The salicylic acid CAS 69-72-7 (SA), salicylic acid 2-O-β-D-glucoside CAS 10366-91-3 (SAG) and salicylic acid acyl glucoside CAS 60517-74-0 (SGE) were separated and quantified using an ultra high-speed high-resolution liquid chromatograph (ACQUITY UPLC H-Class: Waters), a tandem quadrupole mass spectrometer (Xevo TQD, Waters) and a reversed-phase column (ACQUITY UPLC BEH C18, 1.7 μm, 2.1 mm×50 mm: Waters), in negative ion mode. The standard samples used were SA by FujiFilm-Wako and SAG and SGE by Toronto Research Chemicals Co.

Quantitation was by the MRM method, the method being devised using the IntelliStart program of MassLynx™ ver. 4.1 software (Waters). As the mobile phase, there was used 0.1% formic acid (A) and acetonitrile containing 0.1% formic acid (B), with a gradient of 0-1 min: 95% A, 5% B (fixed), 1-8 min: 0% A, 100% B (gradient), 8-10 min: 0% A, 100% B (fixed), 10-11 min: 95% A, 5% B (gradient) and 11-12 min: 95% A, 5% B (fixed), a flow rate of 0.4 ml/min, a column temperature of 40° C. and a sample injection rate of 2 μl. MRM transition for SA was m/z 136.872>93.0 and MRM transition for SAG and SGE was 299.03>136.88, with SAG and SGE being distinguished by difference in retention time.

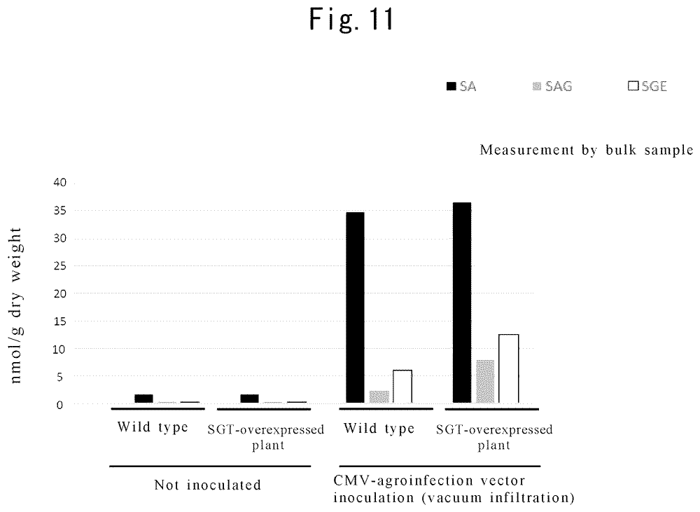

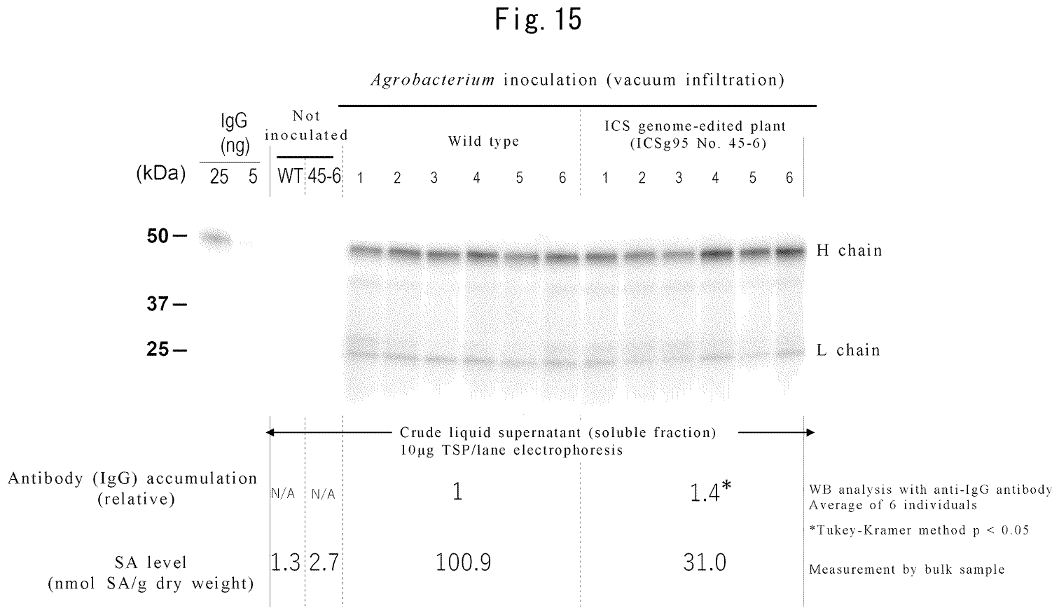

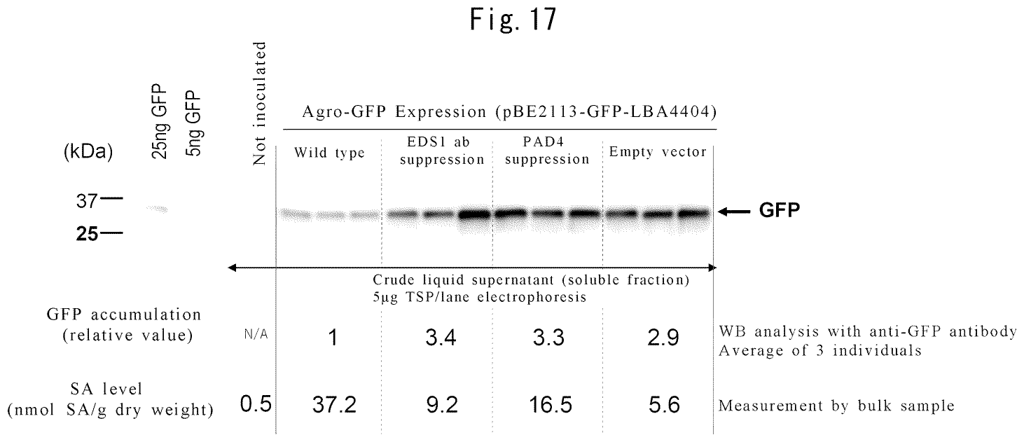

The results of measuring the leaf contents of salicylic acid (SA) and SA metabolites (SAG and SGE), in recombinant plants obtained by manipulating genes associated with the SA biosynthetic pathway and with SA degradation by the methods of Example 20 and Example 21, are shown for the PAL-suppressed plant in FIG. 7, for the ICS genome-edited plant in FIG. 15, and for the EDS1-suppressed plant and PAD4-suppressed plant in FIG. 17. It was confirmed that SA levels had been reduced in the plants compared to the wild type plant. In other words, it was shown that suppression of a gene associated with SA biosynthesis, genome editing or VIGS suppression reduces accumulated amounts of SA in these plants. FIG. 11 shows results of measuring contents of salicylic acid (SA) and SA metabolites (SAG and SGE) in the SGT-overexpressing plant and wild type plant shown in FIG. 10. In the (Agrobacterium) non-inoculated individual, almost no SA or SA metabolites were detected, whereas SA and SA metabolites were detected in the wild type plant inoculated with Agrobacterium (CMV-agroinfection vector) and the SGT-overexpressing plant. In the inoculated SGT-overexpressing plant, accumulated amounts of SAG and SGE were increased compared to the inoculated wild type plant. It was conjectured that accumulation of SAG and SGE, as degradation (metabolism) products of leaf SA, increased as a result of high expression of SGT.

### [Example 22] Genome Editing Vector Assembly for NPR Gene

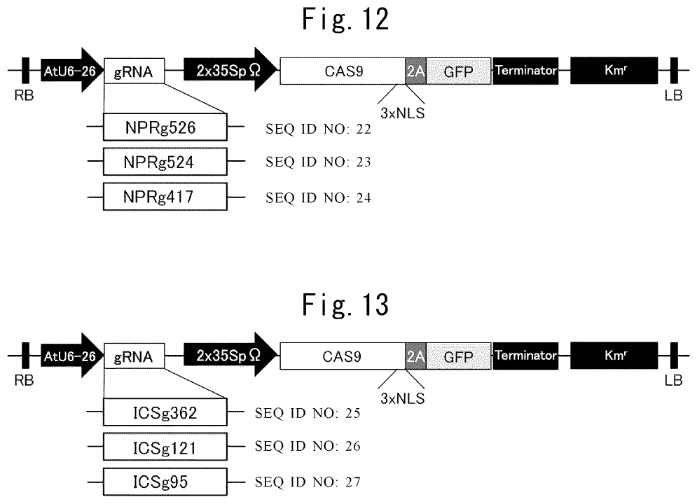

Guide RNAs were designed having simultaneous knockout of the NPRa and NPRb genes, and oligo DNA was synthesized to contain each designed guide RNA target sequence (SEQ ID NOs: 22, 23 and 24). After annealing the sense guide RNA and antisense guide RNA, each oligo DNA was reacted by Golden Gate cloning, and inserted downstream from the guide RNA expression promoter of the genome editing vector pEgP237-2A-GFP (Ueta et al., Scientific Reports, 7:1-8, 2017). E. coli was transformed and the sequences of the obtained clones were confirmed. FIG. 12 schematically shows the structure of a plant expression vector (pEgP237-2A-GFP-NPRgRNA) for guide RNA expression. The vector was introduced into Agrobacterium by the method described in Example 2.

Guide RNA Sequence (oligo DNA+PAM Sequence)

SEQ ID NO: 22:
TGCAGATGTTGCTAAGAGAGGGG (23 mer)
SEQ ID NO: 23:
ACTGCAGATGTTGCTAAGAGAGG (23 mer)
SEQ ID NO: 24:
ACCGATTCACGAGCAGAACTTGG (23 mer)

### [Example 23] Genome Editing Vector Assembly for ICS Gene

Guide RNAs were designed having simultaneous knockout of the ICSa and ICSb genes, and oligo DNA was synthesized to contain each designed guide RNA target sequence (SEQ ID NOs: 25, 26 and 27). After annealing the sense guide RNA and antisense guide RNA, each oligo DNA was reacted by Golden Gate cloning, and inserted downstream from the guide RNA expression promoter of the genome editing vector pEgP237-2A-GFP (Ueta et al., Scientific Reports, 7:1-8, 2017). E. coli was transformed and the sequences of the obtained clones were confirmed. FIG. 13 schematically shows the structure of a plant expression vector (pEgP237-2A-GFP-NPRgRNA) for guide RNA expression. The vector was introduced into Agrobacterium by the method described in Example 2.

### Guide RNA Sequence (Oligo DNA+PAM Sequence)

SEQ ID NO: 25:
GGAGGCAAGAATACTCCCACGTC (23 mer) antisense strand
SEQ ID NO: 26:
ACGATTGGCGTGCTATACGCAGG (23 mer)
SEQ ID NO: 27:
GGAAACTGCTAACCGCACGATAT (23 mer) antisense strand

### [Example 24] Creation of Genome-Edited Plants

Transformation in Nicotiana benthamiana was carried out by the leaf disc method LBA4404 (Horsch R B. et al., (1984), Science, 223:496-498) using Agrobacterium tumefaciens. Leaf discs with diameters of about 1 cm were cut out from tobacco leaves and immersed in LBA4404 cell solution having the guide RNA sequence-inserted pEgP237-2A-GFP (Ueta et al., Scientific Reports, 7:1-8, 2017), and co-culturing was carried out on MS (Murashige-Skoog) agar medium for 2 days. After 3 days, the adhered Agrobacterium was washed off from the co-cultured leaf discs, and the washed co-cultured leaf discs were subcultured in MS solid medium (containing 3% sucrose) with addition of 1 mg/l BAP (6-benzylaminopurine), 0.1 mg/l NAA (naphthaleneacetic acid), 50 mg/l kanamycin and 500 mg/l carbenicillin. After inducing shoots, roots were induced with MS solid medium (containing 3% sucrose) with addition of 50 mg/l kanamycin and 500 mg/l carbenicillin, to obtain multiple lines of cultured plants. The lines of these plants were selected by the methods described in Example 25 and Example 26, and then the cultured plants of the selected lines were conditioned in a closed recombinant greenhouse and grown by soil cultivation to obtain next-generation seeds (T1-T4 individuals).

### [Example 25] Mutation Analysis of NPR Genome-Edited Plants

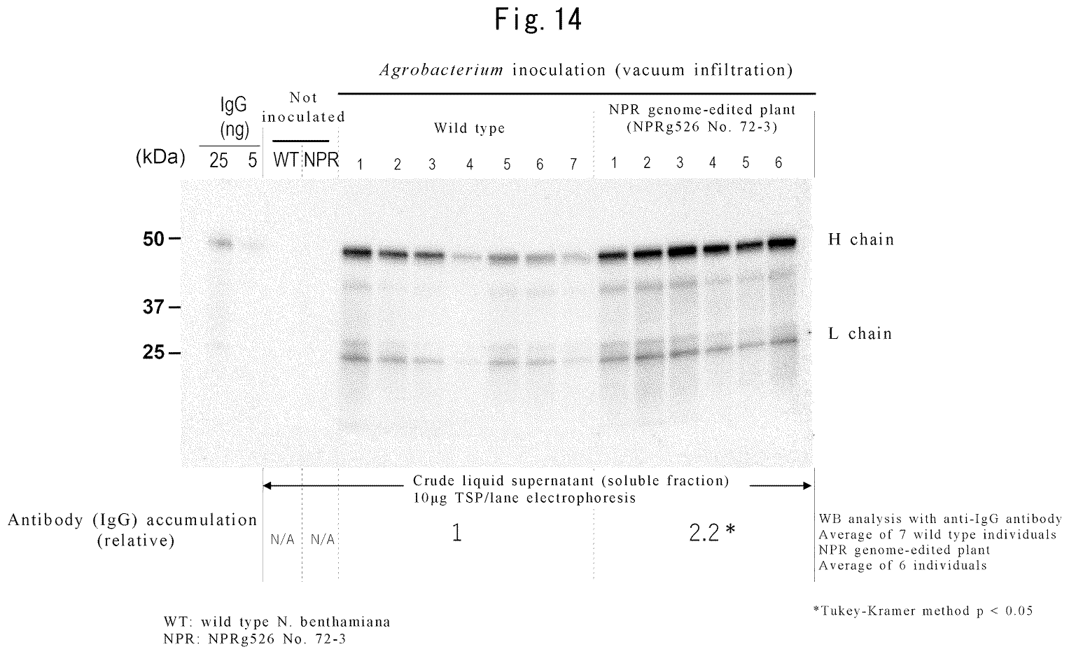

Genomic DNA was extracted from the leaves of candidate genome-edited plants created in Example 24. A MagExtractor-PlantGenome (NPK-501 by Toyobo, Ltd.) was used for the genome extraction, according to the manufacturer's manual. Using the obtained genomic DNA as template, the NPR gene region expected to have the mutation transfer was amplified by PCR using primers (SEQ ID NOs: 28 to 36), and then analyzed by NGS and Sanger sequencing to confirm mutation transfer. The following mutations were assumed to have been introduced into the genome-edited plants that were obtained using the plant expression vector with the guide RNA of SEQ ID NO: 22 inserted (NPRg526 Nos. 72 to 73) (FIG. 14), among the plant expression vectors shown in FIG. 12.

NPRa: Having the following mutant sequence in the homozygous state, based on sequence analysis.

Wild type:
(SEQ ID NO: 55)
TGCAGATGTTGCTAAGAGAG
Mutant:
(SEQ ID NO: 56)
TGCAGATGTTGCTAAG-GAG (A deleted)

NPRb: Having the following mutant sequence in the homozygous state, based on sequence analysis.

Wild type:
(SEQ ID NO: 57)
TGCAGATGTTGCTAAGAGAG
Mutant:
(SEQ ID NO: 58)
TGCAGATGTTGCTAAGATGAG (T inserted)

The results clearly showed that plans with a mutation introduced into the NPR gene (NPR genome-edited plant) had been obtained.

These were used for transient expression of a target protein (IgG). Transient expression was carried out by the same method as described in Example 15 except for using the IgG gene instead of the GFP gene, and the inoculated plants were cultivated under the same conditions as described in Example 16. IgG gene expression levels were evaluated by the method described in Example 27 below.

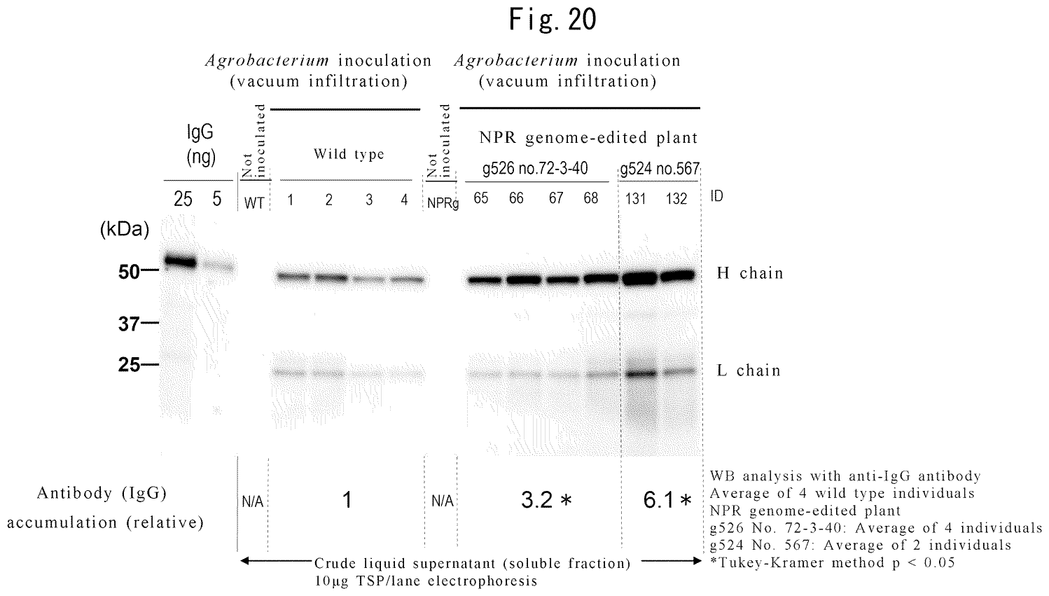

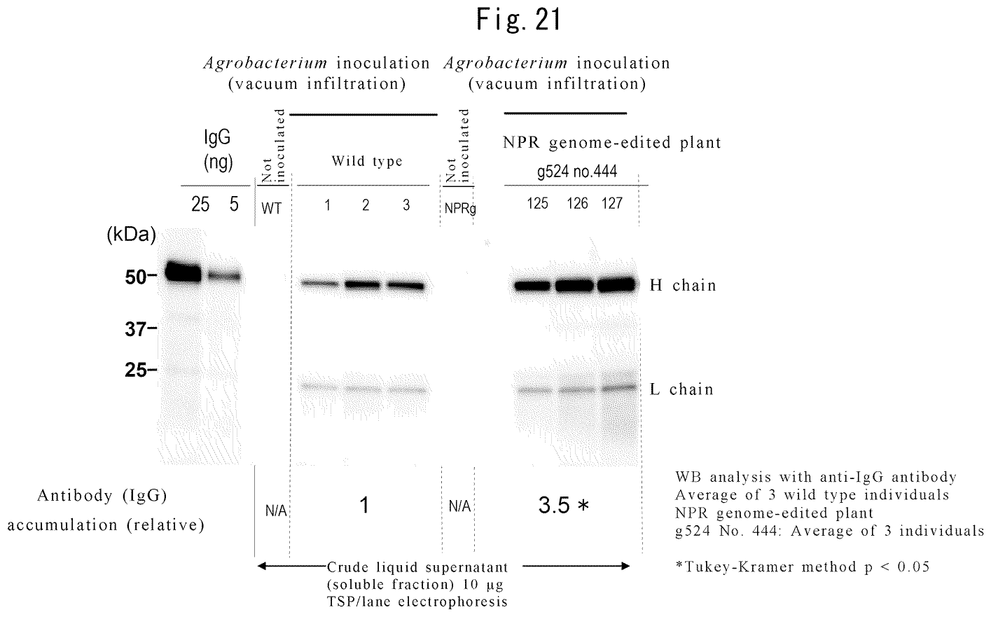

As a result, IgG expression was found to be significantly increased in multiple created lines of NPR genome-edited plants, compared to the wild type plant (FIG. 14, FIG. 20 and FIG. 21). This effect was the same in genetically fixed genome-edited plants (i.e., plants where the plant expression vector had been separated by a genetic method and was not present), with expression of the target protein confirmed to be significantly increased compared to the wild type plant (FIG. 20).

### Mutation Transferred Region Amplification PCR Primers for NGS Analysis

### NPRa Mutation Detection Primers

SEQ ID NO: 28:
acactctttccctacacgacgctcttccgatctCTAATGGTCCATTGCATATTTC (55 mer)
(forward)
SEQ ID NO: 29:
gtgactggagttcagacgtgtgctcttccgatctGTGCAAGATCTAGAAGTTCTG (55 mer)
(reverse)

### NPRb Mutation Detection Primers

SEQ ID NO: 30:
acactctttccctacacgacgctcttccgatctGTTCCACTTCACATT
CCTTC (54 mer) (forward)

SEQ ID NO: 29: Same as above

### Mutation Transferred Region Amplification PCR Primers for Sanger Sequencing

### NPRa Mutation Detection Primers

SEQ ID NO: 31:
CAGAGCAACCCCTCTTGTCTGTAC (24 mer) (forward)
SEQ ID NO: 32:
CAAACTAAGTGGAAGATCATACAG (24 mer) (reverse)

### NPRb Mutation Detection Primers

SEQ ID NO: 33:
GTCATTCAAGGTAAGTTTCTAGTG (24 mer) (forward)
SEQ ID NO: 34:
CTTATGGAAGATCGTATACAGTG (23 mer) (reverse)

### Sanger Sequencing Primers

### For NPRa Sequencing

SEQ ID NO: 35:
CTAATGGTCCATTGCATATTTC (22 mer)

### For NPRb Sequencing

SEQ ID NO: 36:
GTTCCACTTCACATTTCCTTC (21 mer)

### [Example 26] Mutation Analysis of ICS Genome-Edited Plants

Genomic DNA was extracted from the leaves of candidate genome-edited plants created in Example 24. A MagExtractor-PlantGenome (NPK-501 by Toyobo, Ltd.) was used for the genome extraction, according to the manufacturer's manual. Using the obtained genomic DNA as template, the ICS gene region expected to have the mutation transfer was amplified by PCR using primers (SEQ ID NOs: 36 to 43), and then used in next-generation sequencing (NGS) analysis and Sanger sequence analysis to confirm mutation transfer. The following mutations were assumed to have been introduced into the genome-edited plants that were obtained using the plant expression vector with the guide RNA of SEQ ID NO: 27 inserted (ICSg95 Nos. 45 to 46) (FIG. 15), among the plant expression vectors shown in FIG. 13.

ICSa: Having the following mutant sequences in the homozygous state, based on sequence analysis.

### Wild Type:

(SEQ ID NO: 59)
CTTCTAAAGTGGTCAGTGTAGCTGGTGTCGGCTCTGCTGTCTTCTTTAC
TCATTTACGCCCTTTTTCCTTTGACGATTGGCGTGCTATA (the
underline indicates ICSg95 sequence
(complementary strand)).
Mutant:
CTTCTAAAGTG--------------------------------------

CTATA (SEQ ID NO: 60) (“-” indicates that the nucleotides at those positions are deleted. Total: 73 nucleotide deletions.)
ICSb: Having the following wild type and mutant sequences in the heterozygous state, based on sequence analysis.

Wild type:
(SEQ ID NO: 61)
TTGACGATTGGCGTGCTATA

Mutant: TTGA-----GGCGTGCTATA (SEQ ID NO: 62) (“-” indicates that the nucleotides at those positions are deleted. Total: 5 nucleotide deletions.)

The results clearly showed that mutation-introduced plants (ICS genome-edited plants) were successfully obtained.

These were used for transient expression of a target protein (IgG). Transient expression was carried out by the same method as described in Example 15 except for using the IgG gene instead of the GFP gene, and the inoculated plants were cultivated under the same conditions as described in Example 16. IgG gene expression levels were evaluated by the method described in Example 27 below.

As a result, IgG expression was found to be significantly increased in the ICS genome-edited plants, compared to the wild type plant (FIG. 15). In addition, when SA in the plants was measured by the method described in Example 21, SA accumulation was found to be reduced in the ICS genome-edited plants compared to the wild type plant (FIG. 15). In other words, it was shown that mutation of an enzyme gene associated with SA biosynthesis by genome editing reduces accumulated amounts of SA in these plants.

### Mutation Transferred Region Amplification PCR Primers for NGS Analysis

### ICSa Mutation Detection Primers

SEQ ID NO: 37:
acactctttccctacacgacgctcttccgatctGTCGAAGGGCAGCTGCTGATTC (5 5mer)
(forward)
SEQ ID NO: 38:
gtgactggagttcagacgtgtgctcttccgatctTTTAGTTGCCCAAATCTTCAG (5 5mer)
(reverse)
ICSb mutation detection primers
SEQ ID NO: 39:
acactctttccctacacgacgctcttccgatctGGTCGAAGGCCAGCTGTTGAAC (55 mer)
(forward)
SEQ ID NO: 40:
gtgactggagttcagacgtgtgctcttccgatctCTACACTAATTTCATCATCTATG
(57 mer) (reverse)

### Mutation Transferred Region Amplification PCR Primers for Sanger Sequencing

### ICSa Mutation Detection Primers

SEQ ID NO: 41:
GTCGAAGGGCAGCTGCTGATTC (22 mer) (forward)
SEQ ID NO: 42:
TTTAGTTGCCCAAATCTTCAG (21 mer) (reverse)

### ICSb Mutation Detection Rimers

SEQ ID NO: 43:
GGTCGAAGGCCAGCTGTTGAAC (22 mer) (forward)
SEQ ID NO: 44:
CTACACTAATTTCATCATCTATG (23 mer) (reverse)

### Sanger Sequencing Primers

### For ICSa Sequencing

SEQ ID NO: 42: Same as above

### For ICSb Sequencing

SEQ ID NO: 44: Same as above

### [Example 27] Western Blotting for Detection of Target Protein (IgG) in Inoculated Plants

After harvesting the leaves from IgG gene-inoculated plants and crushing them using liquid nitrogen, PBS buffer was added and the crushed leaves were ground. The ground solution was centrifuged (15,000 rpm, 4° C., 10 min), and then the crude supernatant was collected. The total amount of protein (TSP) was quantified by the BCA method, and for Western blot analysis, each lane was adjusted to a fixed amount of TSP, electrophoresed in 12% SDS-PAGE gel, and then transferred to a PVDF membrane. Immunostaining carried out with a specific antibody (anti-IgG antibody) was followed by chemiluminescence detection using ChemiDoc (Bio-Rad), for quantification of the band strength.

### [Example 28] Transient Gene Suppression of EDS1 Gene

The EDS1a gene (Niben101Scf06720g01024) and EDS1b gene (Niben101Scf02237g01002) of Nicotiana benthamiana were cloned, and a partial sequence of 300 bp (SEQ ID NO: 45) which is able to simultaneously target both was introduced into a viral vector and suppression of EDS1 gene expression was induced by the VIGS method. For analysis of expression level of the EDS1 gene, a region allowing simultaneous detection of the EDS1a gene and EDS1b gene was evaluated by quantitative RT-PCR. A target protein (GFP) was expressed in the plants by agroinfiltration (vacuum infiltration), inoculated plants were cultivated by the method described in Example 16, and GFP was then detected by the method described in Example 17.

As a result of examining gene suppression in the plants by VIGS using quantitative RT-PCR, it was confirmed that the expression level of the EDS1 gene was 0.67 in tobacco inoculated with an empty vector containing no EDS1 gene, while the expression level of the EDS1 gene was 0.18 in the EDS1 expression-suppressed tobacco created in Example 28, where the expression level of the EDS1 gene in the wild type plant (without gene expression suppression) was defined as 1.

As a result of evaluating GFP expression levels in the plants by Western blot analysis (FIG. 17), GFP expression was confirmed to be increased in the EDS1-suppressed plants compared to the wild type plant and the empty vector inoculated plant. The results of measuring the leaf contents of salicylic acid (SA) and SA metabolites (SAG and SGE) in EDS1 expression-suppressed tobacco by the methods described in Example 20 and Example 21 are shown at bottom in FIG. 17. It was confirmed that SA levels had been reduced in the plants compared to the wild type plant. This indicates that VIGS suppression of an EDS gene associated with SA biosynthesis reduces accumulated amounts of SA in these plants.

SEQ ID NO: 45: EDS1 gene partial sequence of 300 bp (the following sequence is an antisense sequence as it was introduced into the antisense strand)

GTTTCTTAGTTCCTCCACTTCTGCCCAAAAACAAGACTCAGAGCGTTCA
CCTGTTTGCACCCTCTCTTCATGCTCTAACCATCGTTGTGTGAACCTAT
AACGCTTCGGCCTAGCCCTGATCATGTAAGGTCCTGTATCTTCATTCTT
CAAATGCCTGTAATAGTTTGCAATATCCAAGGGCTCAACTTGCCTGCGG
AACTGCGTCCCTAGTTTTATCCATTCCTTTCTTCCCTCAAAACTATCTG
GGAGCTCATACCTTTTCAACATTTCAATGATTTCGTCCCATATTCCTGC
TAGCTC

### [Example 29] Transient Gene Suppression of PAD4 Gene

The PAD4 gene (Niben101Scf02544g01012) of Nicotiana benthamiana was cloned, part of the sequence (300 bp) was introduced into a viral vector, and suppression of PAD4 gene expression was induced by the VIGS method. The PAD4 gene expression suppressing effect was evaluated by quantitative RT-PCR (FIG. 16). A target protein (GFP) was expressed in the plants by agroinfiltration (vacuum infiltration), inoculated plants were cultivated by the method described in Example 16, and GFP was then detected by the method described in Example 17.

As a result of examining gene suppression in the plants by VIGS using quantitative RT-PCR, it was confirmed that the expression level of the PAD4 gene was 0.79 in tobacco inoculated with an empty vector containing no PAD4 gene, while the expression level of the PAD4 gene was 0.2 in the PAD4 expression-suppressed tobacco created in Example 29, where the expression level of the PAD4 gene in the wild type plant (without gene expression suppression) was defined as 1.

As a result of evaluating GFP expression levels in the plants by Western blot analysis (FIG. 17), GFP expression was confirmed to be increased in the PAD4-suppressed plants compared to the wild type plant and the empty vector inoculated plant. The results of measuring the leaf contents of salicylic acid (SA) and SA metabolites (SAG and SGE) in PAD4 expression-suppressed tobacco by the methods described in Example 20 and Example 21 are shown at bottom in FIG. 17. It was confirmed that SA levels had been reduced in the plants compared to the wild type plant. This indicates that VIGS suppression of a PAD4 gene associated with SA biosynthesis reduces accumulated amounts of SA in these plants.

SEQ ID NO: 46: PAD4 gene partial sequence of 300 bp (the following sequence is an antisense sequence as it was introduced into the antisense strand)

TCATAGCTTGCCTCAGGTAGATTGCCTCCCATGAAGCTTCTAATCTCTA
TAAATTGCCAATGAACTTTCTGAATAAAATGCGTATAACCAAGATGATC
CTCGAGGCTAGAACTTGGTGAACCATTCAGTAACGTCAAGTAAAGTAAC
TTAACGATAAGCATCCCATTATCGACACAAACTGCACCCATGTTGGTGC
AGAACAAGTAGCTCCCAAAGGGCCAAAATGAACTCTTACATTCACCTTT
TGATATAACTTCAAGAGAAGCCAACACAACGCGGAATAGCTGAGTCTTG
TTTTCC

### [Example 30] Genome Editing Vector Assembly for EDS Gene

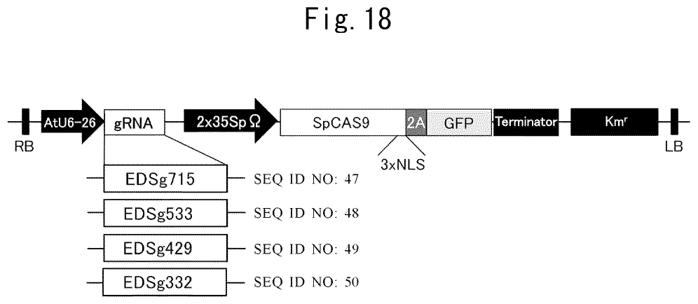

Guide RNAs were designed having simultaneous knockout of the EDS1 gene (Niben101Scf06720g01024) and EDS1b gene (Niben101Scf02237g01002), and oligo DNA was synthesized to contain each designed guide RNA target sequence (SEQ ID NOs: 47, 48, 49 and 50). After annealing the sense guide RNA and antisense guide RNA, each oligo DNA was reacted by Golden Gate cloning, and inserted downstream from the guide RNA expression promoter of the genome editing vector pEgP237-2A-GFP (Ueta et al., Scientific Reports, 7:1-8, 2017). E. coli was transformed and the sequences of the obtained clones were confirmed. FIG. 18 schematically shows the structure of a plant expression vector (pEgP237-2A-GFP-EDSgRNA) for guide RNA expression.

### Guide RNA Sequence (Oligo DNA+PAM Sequence)

SEQ ID NO: 47:
GCAATGGCATTTGAAGACAAGGG (23 mer)
SEQ ID NO: 48:
CACTGGAAATGGGAAACTGGTGG (23 mer)
SEQ ID NO: 49:
TATGCTGCATGTAATCTGAAAGG (23 mer)
SEQ ID NO: 50:
ATCCCGGAATTATCAGCACGAGG (23 mer)

### [Example 31] Quantitative Analysis of PR1 Gene Expression

After liquid nitrogen freezing of fresh leaves of the agroinfiltration-inoculated plant individuals of Example 15 using the NPR-suppressed plants created in Example 3, they were ground, and the total RNA was extracted by AGPC extraction (Chomczynski, P. et al., (1987), Anal. Biochem. 162:156-159). The total RNA was subjected to DNase treatment and cDNA was synthesized by reverse transcription using random primers, and then real-time PCR was carried out using a LightCycler 96 system (Roche Diagnostics). Primers (SEQ ID NOs: 51 and 52) and SYBR Green I were used to detect the PR1 gene ((Niben101Scf13926g01014). Nb EF1α was used as an internal standard for quantification. Primers (SEQ ID NOs: 53 and 54) and SYBR Green I were used to detect the EF1α gene.

As a result, the expression level of the PR1 gene was reduced to 0.33 in the IR-NPR-transformed benthamiana line No. 144, and to 0.54 in line No. 168, where the gene expression level in the wild type was defined as 1 (data not shown). The results suggest that suppressed expression of NPR (SA receptor) resulted in inhibition of SA signal transduction and suppression of expression of the gene associated with the defensive response (PR gene), which is expressed after NPR-mediated signal transduction.

### PR1 Gene Detection Primers

SEQ ID NO: 51:
TCGTGCAGTTGTAGGCGTAG (20 mer) (forward)
SEQ ID NO: 52:
TGTGCATAGGCTGCTACCTG (20 mer) (reverse)

### EF1α Gene Detection Primers

SEQ ID NO: 53:
TGGACACAGGGACTTCATCA (20 mer) (forward)
SEQ ID NO: 54:
CGGGTCTGTCCATCCTTAGA (20 mer) (reverse)

### INDUSTRIAL APPLICABILITY

The present invention can be utilized as a method for increasing production of useful substances using plans having suppression of inherent resistance mechanisms.

1. A method for creating a plant with suppressed resistance to a pathogen, wherein the method comprises suppressing expression of a gene associated with a resistance mechanism in the plant or disrupting the gene.

2. The method according to claim 1, wherein the gene associated with a resistance mechanism is an NPR gene (nonexpressor of pathogenesis-related genes) or an isochorismate synthase (ICS) gene.

3. The method according to claim 2, the method comprising:
(i) a step of preparing a nucleic acid construct for suppression expression of the gene associated with a resistance mechanism or disruption of the gene,
(ii) a step of transferring the nucleic acid construct into plant cells or a plant tissue, and
(iii) a step of culturing the plant cells or the plant tissue having the transferred nucleic acid construct to create a plant expressing the nucleic acid construct.

4. The method according to claim 3, wherein the nucleic acid construct is a first nucleic acid construct comprising a nucleic acid sequence complementary to an mRNA of the gene associated with a resistance mechanism, or a transcriptional product thereof.

5. The method according to claim 4, wherein in the first nucleic acid construct, the nucleic acid sequence complementary to the mRNA of the gene associated with a resistance mechanism, or a transcriptional product thereof is selected from the group consisting of an antisense RNA, an RNA interference (RNAi) molecule, and a virus-induced gene silencing (VIGS) molecule.

6. The method according to claim 2, the method comprising:
(i) a step of preparing a second nucleic acid construct comprising a nucleic acid sequence encoding a genome editing-related protein with a target site in the gene associated with a resistance mechanism,
(ii) a step of transferring the nucleic acid construct into plant cells or a plant tissue, and
(iii) a step of culturing the plant cells or the plant tissue having the transferred nucleic acid construct to create a plant with a mutation transferred into the gene sequence.

7. The method according to claim 6, wherein the genome editing-related protein is selected from the group consisting of a Cas protein, a zinc finger nuclease, and a TAL effector nuclease.

8. The method according to claim 3, wherein the transfer of the nucleic acid construct into the plant cells or the plant tissue in step (ii) is carried out using a transient expression system.

9. The method according to claim 8, wherein the transient expression system is selected from agroinfiltration, a plant viral vector, and agroinfection that combines agroinfiltration and a plant viral vector.

10. The method according to claim 9, wherein the plant viral vector is a full viral vector or a deconstructed viral vector.

11. The method according to claim 9, wherein the agroinfection is a combination of a CMV vector and an Agrobacterium T-DNA.

12. The method according to claim 3, wherein the transfer of the nucleic acid construct into the plant cells or the plant tissue in step (ii) is carried out by permeation or injection of a solution containing the nucleic acid construct into the plant cells or the plant tissue.

13-23. (canceled)

24. Transformed plant cells, a transgenic plant, or a genome-edited plant with suppressed expression of a gene associated with a resistance mechanism or disruption of the gene.

25. The transformed plant cells, the transgenic plant, or the genome-edited plant according to claim 24, wherein the gene associated with a resistance mechanism is an NPR gene (nonexpressor of pathogenesis-related genes) or an isochorismate synthase (ICS) gene.

26-29. (canceled)

30. A method for producing a target protein using a plant, wherein the method comprises:
transferring into the transformed plant cells, the transgenic plant, or the genome-edited plant according to claim 24, a construct for expression of the target protein in the plant,
thereby allowing the expression of the construct in the transformed plant cells, the transgenic plant, or the genome-edited plant.

31. (canceled)

32. A method for producing a target protein using a plant, wherein the method comprises:
transferring into the transformed plant cells, the transgenic plant, or the genome-edited plant according to claim 25, a construct for expression of the target protein in the plant,
thereby allowing the expression of the construct in the transformed plant cells, the transgenic plant, or the genome-edited plant.

33. The method according to claim 9, wherein the plant viral vector is obtained from tobacco mosaic virus (TMV), plum pox virus (PPV), turnip vein-clearing virus (TVCV), potato virus X (PVX), bean yellow dwarf virus (BEYDV), alfalfa mosaic virus (AIMV), cucumber mosaic virus (CMV), cowpea mosaic virus (CPMV), zucchini yellow mosaic virus (ZYMV), tobacco rattle virus (TRV), apple latent spherical virus (ACMV), bromo mosaic virus (BMV), tomato mosaic virus (ToMV), tomato yellow leaf curl virus (TYLCV), or tomato golden mosaic virus (TGMV).
